(19) United States
(12) Patent Application Publication
(10) Pub. No.: US 2024/0398209 A1
(43) Pub. Date: Dec. 5, 2024

(54) IMAGE PROCESSING APPARATUS, TREATMENT SYSTEM, LEARNING APPARATUS, AND IMAGE PROCESSING METHOD

(71) Applicant: Olympus Corporation, Tokyo (JP)

(72) Inventors: Hiroshi Suzuki, Tokyo (JP); Koichiro Watanabe, Tokyo (JP)

(21) Appl. No.: 18/800,816

(22) Filed: Aug. 12, 2024

Related U.S. Application Data
(63) Continuation of application No. PCT/JP2022/011119, filed on Mar. 11, 2022.

(51) Int. Cl. A61B 1/00; A61B 1/04; A61B 17/32; G06V 10/774; G06V 20/50; G06V 20/70; G16H 30/40

(52) U.S. Cl. CPC A61B 1/000096; A61B 1/044; A61B 1/046; A61B 17/320016; A61B 17/320068; G06V 10/774; G06V 20/50; G06V 20/70; G16H 30/40; G06V 2201/03

(57) ABSTRACT

An image processing apparatus includes a processor including hardware, the processor being configured to: estimate a target object in turbidity image data including turbidity generated when a living body is treated by an energy treatment instrument, from the turbidity image data that is input, using a learned model obtained by performing machine learning using teacher data obtained by associating annotation image data with an identification result of identifying the target object in the turbidity image data, the annotation image data having an annotation applied to the target object; and generate a display image related to the target object based on the turbidity image data that is input and the estimated target object.

### CROSS-REFERENCE TO RELATED APPLICATION

This application is a continuation of International Application No. PCT/JP2022/011119, filed on Mar. 11, 2022, the entire contents of which are incorporated herein by reference.

### BACKGROUND

### 1. Technical Field

The present disclosure relates to an image processing apparatus, a treatment system, a learning apparatus, and an image processing method.

### 2. Related Art

In arthroscopic surgery, there is known a technique in which the inside of a joint is inflated with a perfusate such as physiological saline by a perfusion device to secure a visual field, and treatment by a treatment portion is performed (for example, refer to JP 4564595 B2). In such technique, bone meal and spinal fluid, that are bone shavings, are generated by crushing the bone by a hammering operation of an ultrasound treatment instrument, and the bone meal and the spinal fluid are delivered out from a visual field of an endoscope by the perfusate, thereby securing a visual field for the treatment portion.

### SUMMARY

In some embodiments, an image processing apparatus includes a processor including hardware, the processor being configured to: estimate a target object in turbidity image data including turbidity generated when a living body is treated by an energy treatment instrument, from the turbidity image data that is input, using a learned model obtained by performing machine learning using teacher data obtained by associating annotation image data with an identification result of identifying the target object in the turbidity image data, the annotation image data having an annotation applied to the target object; and generate a display image related to the target object based on the turbidity image data that is input and the estimated target object.

In some embodiments, a treatment system includes: an energy treatment instrument; an imaging device; and an image processing apparatus, wherein the energy treatment instrument includes a treatment instrument main body portion extending from a proximal end side to a distal end side in a longitudinal direction of the energy treatment instrument, and a treatment portion provided on a distal end side of the treatment instrument main body portion, the treatment portion being configured to treat a living body, the imaging device includes a casing main body configured to be inserted into a subject, the casing extending from a proximal end side to a distal end side in a longitudinal direction of the imaging device, an illumination portion configured to emit illumination light toward at least an area in which the living body is treated by the energy treatment instrument, and an imaging portion configured to generate turbidity image data including at least a part of an area in which the living body is treated by the energy treatment instrument and turbidity is generated, and the image processing apparatus includes a processor including hardware, the processor being configured to: estimate a target object in the turbidity image data including the turbidity generated when the living body is treated by the energy treatment instrument, from the turbidity image data that is input, using a learned model obtained by performing machine learning using teacher data obtained by associating annotation image data with an identification result of identifying the target object in the turbidity image data, the annotation image data having an annotation applied to the target object; and generate a display image related to the target object based on the turbidity image data that is input and the estimated target object.

In some embodiments, a learning apparatus includes a processor including hardware, the processor being configured to generate a learned model by performing machine learning using teacher data, wherein the teacher data uses, as input data, a plurality of pieces of treatment image data obtained by capturing an image of an area in which a living body is treated by an energy treatment instrument and a plurality of pieces of annotation image data to which an annotation of a target object included in a plurality of treatment images respectively corresponding to the plurality of pieces of treatment image data is applied, and outputs, as output data, an identification result in which the target object is identified, the target object being included in an image corresponding to image data including at least a part of the area in which the living body is treated by the energy treatment instrument.

In some embodiments, provided is an image processing method executed by an image processing apparatus including a processor including hardware. The method includes: estimating a target object in turbidity image data including turbidity generated when a living body is treated by an energy treatment instrument, from the turbidity image data that is input, using a learned model obtained by performing machine learning using teacher data obtained by associating annotation image data with an identification result of identifying the target object in the turbidity image data, the annotation image data having an annotation applied to the target object; and generating a display image related to the target object based on the turbidity image data that is input and the estimated target object.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of a treatment system according to an embodiment of the present disclosure;

FIG. 2 is a diagram illustrating a state in which a bone hole is formed by an ultrasound probe according to the embodiment of the present disclosure;

FIG. 3A is a schematic diagram illustrating a schematic configuration of the ultrasound probe according to the embodiment of the present disclosure;

FIG. 3B is a schematic diagram in a direction of an arrow A in FIG. 3A;

FIG. 4 is a block diagram illustrating an outline of a functional configuration of the entire treatment system according to the embodiment of the present disclosure;

FIG. 5 is a block diagram illustrating a detailed functional configuration of an endoscope device according to the embodiment of the present disclosure;

FIG. 6A is a diagram illustrating a state in which a visual field of an endoscope according to the embodiment of the present disclosure is good;

FIG. 6B is a diagram illustrating a state in which the visual field of the endoscope according to the embodiment of the present disclosure is poor;

FIG. 7 is a block diagram illustrating a detailed functional configuration of a processing apparatus according to the embodiment of the present disclosure;

FIG. 8 is a block diagram illustrating a detailed functional configuration of a perfusion device according to the embodiment of the present disclosure;

FIG. 9 is a block diagram illustrating a detailed functional configuration of an illumination device according to the embodiment of the present disclosure;

FIG. 10 is a schematic diagram illustrating a schematic configuration of the illumination device according to the embodiment of the present disclosure;

FIG. 11 is a diagram illustrating a relationship between transmission characteristics and wavelength bands of a red filter, a green filter, and a blue filter according to the embodiment of the present disclosure;

FIG. 12 is a diagram illustrating a relationship between a transmission characteristic and a wavelength band of an IR transmission filter according to the embodiment of the present disclosure;

FIG. 13 is a block diagram illustrating a detailed functional configuration of an image processor according to the embodiment of the present disclosure;

FIG. 14 is a block diagram schematically illustrating exchange of a part of signals configuring the image processor according to the embodiment of the present disclosure;

FIG. 15 is a block diagram illustrating a detailed functional configuration of a turbidity correction unit according to the embodiment of the present disclosure;

FIG. 16 is a flowchart illustrating an outline of treatment performed by an operator using the treatment system according to the embodiment of the present disclosure;

FIG. 17 is a diagram illustrating an outline of processing executed in cutting treatment by an endoscope control device according to the embodiment of the present disclosure;

FIG. 18 is a diagram illustrating an example of a first image generated by a first image generation unit according to the embodiment of the present disclosure;

FIG. 19 is a diagram illustrating an example of a second image generated by a second image generation unit according to the embodiment of the present disclosure;

FIG. 20 is a diagram schematically illustrating an estimation result of a target object estimated by an estimation unit according to the embodiment of the present disclosure;

FIG. 21 is a diagram illustrating an example of a display image generated by a display image generation unit according to the embodiment of the present disclosure;

FIG. 22 is a diagram schematically illustrating a method of generating a learned model generated by a learning unit according to the embodiment of the present disclosure; and

FIG. 23 is a diagram schematically illustrating a method of generating another learned model generated by a learning unit according to a modification of the embodiment of the present disclosure.

### DETAILED DESCRIPTION

Hereinafter, modes for carrying out the present disclosure will be described in detail with reference to the drawings. Note that the present disclosure is not limited to the following embodiments. In addition, each drawing referred to in the following description merely schematically illustrates a shape, a size, and a positional relationship to an extent that a content of the present disclosure can be understood. That is, the present disclosure is not limited only to the shape, the size, and the positional relationship illustrated in each drawing. Furthermore, in the following description, the same portions are denoted by the same reference numerals in the description of the drawings.

### Schematic Configuration of Treatment System

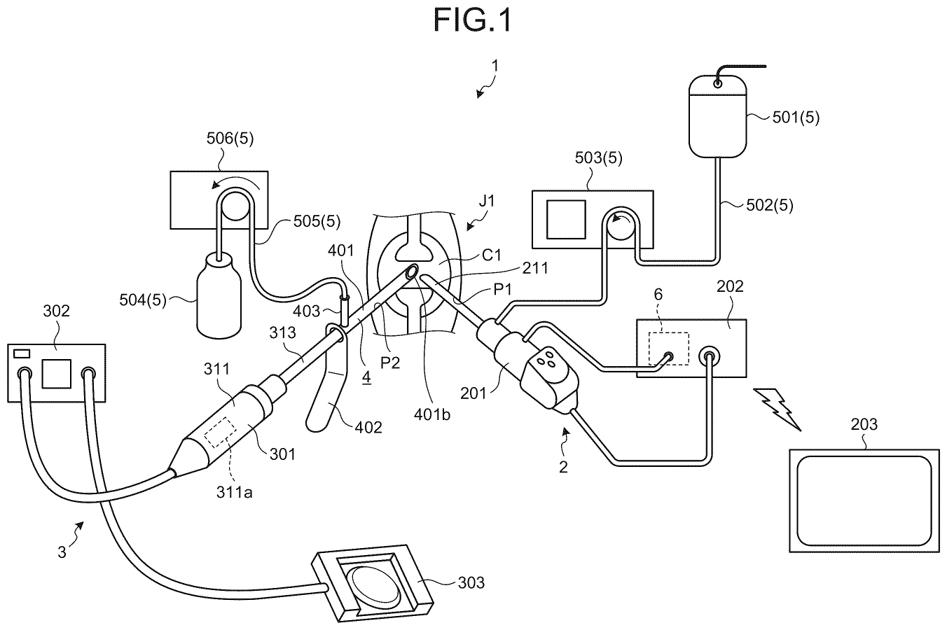

FIG. 1 is a diagram illustrating a schematic configuration of a treatment system 1 according to an embodiment. The treatment system 1 illustrated in FIG. 1 treats a biological tissue such as a bone by applying ultrasound vibration to the biological tissue. Here, the treatment is, for example, removal or cutting of the biological tissue such as bone. Note that FIG. 1 illustrates a treatment system that performs anterior cruciate ligament reconstruction surgery as the treatment system 1.

The treatment system 1 illustrated in FIG. 1 includes an endoscope device 2, a treatment device 3, a guiding device 4, a perfusion device 5, and an illumination device 6.

### Configuration of Endoscope Device

First, a configuration of the endoscope device 2 will be described.

The endoscope device 2 includes an endoscope 201, an endoscope control device 202, and a display device 203.

In the endoscope 201, a distal end portion of an insertion unit 211 is inserted into a joint cavity C1 of a knee joint J1 of a subject through a first portal P1 that allows the inside of the joint cavity C1 and the outside of the skin to communicate with each other. The endoscope 201 irradiates the inside of the joint cavity C1, captures illumination light (subject image) reflected in the joint cavity C1, and captures an image of the subject image to generate image data.

The endoscope control device 202 executes various types of image processing on the image data captured by the endoscope 201, and causes the display device 203 to display a display image corresponding to the image data after the image processing. The endoscope control device 202 is connected to the endoscope 201 and the display device 203 in a wired or wireless manner.

The display device 203 receives, via the endoscope control device 202, data, image data (display image), audio data, and the like transmitted from each device configuring the treatment system 1, and displays, notifies, and outputs the display image according to the received data. The display device 203 is configured using a display panel made of liquid crystal or organic electro-luminescence (EL).

### Configuration of Treatment Device

Next, a configuration of the treatment device 3 will be described.

The treatment device 3 includes a treatment instrument 301, a treatment instrument control device 302, and a foot switch 303.

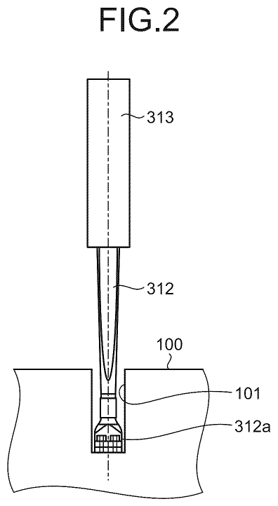

The treatment instrument 301 includes a treatment instrument main body 311, an ultrasound cutting unit 312 (refer to FIG. 2 described later), and a sheath 313.

The treatment instrument main body 311 is formed in a cylindrical shape. In addition, the treatment instrument main body 311 accommodates an ultrasound transducer 312a (refer to FIG. 2 described later) formed by a bolt-clamped Langevin-type transducer and generating ultrasound vibration according to supplied drive power.

The treatment instrument control device 302 supplies drive power to the ultrasound transducer 312a in response with an operation of the foot switch 303 by an operator. Note that the supply of the drive power is not limited to the operation of the foot switch 303, and may be performed, for example, in response to an operation of an operating unit (not illustrated) provided in the treatment instrument 301.

The foot switch 303 is an input interface configured to allow the operator to perform operation with the foot when driving the ultrasound cutting unit 312.

Next, the ultrasound cutting unit 312 will be described.

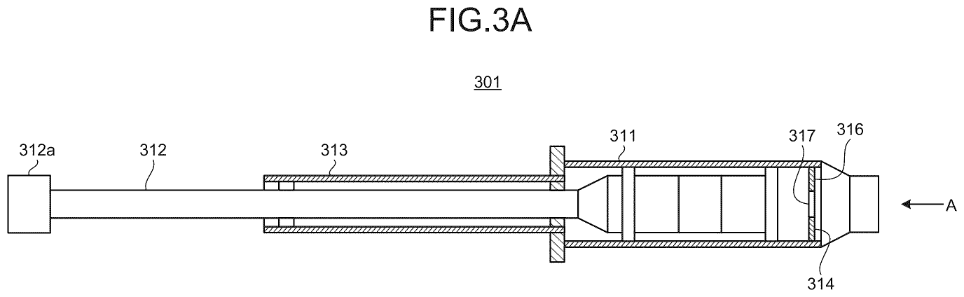

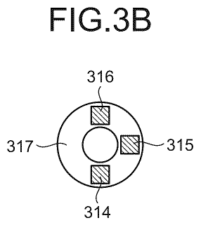

FIG. 2 is a diagram illustrating a state in which a bone hole 101 is formed by the ultrasound cutting unit 312. FIG. 3A is a schematic diagram illustrating a schematic configuration of the ultrasound cutting unit 312. FIG. 3B is a schematic diagram in a direction of an arrow A in FIG. 3A.

As illustrated in FIGS. 2, 3A, and 3B, the ultrasound cutting unit 312 is made of, for example, a titanium alloy or the like, and has a substantially cylindrical shape. In addition, a proximal end portion of the ultrasound cutting unit 312 is connected to the ultrasound transducer 312a in the treatment instrument main body 311. Furthermore, the ultrasound cutting unit 312 transmits the ultrasound vibration generated by the ultrasound transducer 312a from the proximal end to the distal end. Specifically, the ultrasound vibration in the embodiment is longitudinal vibration in the longitudinal direction of the ultrasound cutting unit 312 (vertical direction in FIG. 2). As illustrated in FIG. 2, the ultrasound transducer 312a is provided at the distal end portion of the ultrasound cutting unit 312.

The sheath 313 is formed in a cylindrical shape longer than the treatment instrument main body 311 and covers a part of the outer periphery of the ultrasound cutting unit 312 from the treatment instrument main body 311 to an arbitrary length.

The ultrasound transducer 312a of the ultrasound cutting unit 312 in the treatment instrument 301 configured as such is inserted into the joint cavity C1 while being guided by the guiding device 4 inserted into the joint cavity C1 through a second portal P2 that allows the inside of the joint cavity C1 and the outside of the skin to communicate with each other.

Subsequently, when the treatment instrument 301 generates ultrasound vibration while the ultrasound transducer 312a of the ultrasound cutting unit 312 is in contact with a treatment target region 100 of the bone, a portion of the bone mechanically colliding with the ultrasound transducer 312a is pulverized into fine particles by hammering action (refer to FIG. 2).

Thereafter, when the ultrasound transducer 312a of the ultrasound cutting unit 312 is pushed to the treatment target region 100 by the operator, the treatment instrument 301 enters the inside of the treatment target region 100 while the ultrasound transducer 312a pulverizes the bone. As a result, the bone hole 101 is formed in the treatment target region 100.

In addition, the treatment instrument main body 311 has a circuit board 317 provided at the proximal end thereof, in which the circuit board 317 is mounted with a posture detection unit 314, a central processing unit (CPU) 315, and a memory 316 (refer to FIGS. 3A and 3B).

The posture detection unit 314 includes a sensor that detects rotation and movement of the treatment instrument 301. The posture detection unit 314 detects movement in three axial directions orthogonal to each other including an axis parallel to the longitudinal axis of the ultrasound cutting unit 312 and rotation around each axis. The treatment instrument control device 302 described above determines that the treatment instrument 301 is stationary when a detection result of the posture detection unit 314 does not change for a certain period of time. The posture detection unit 314 includes, for example, a triaxial angular velocity sensor (a gyro sensor), an acceleration sensor, and the like.

The CPU 315 controls the operation of the posture detection unit 314 and transmits and receives information to and from the treatment instrument control device 302. The CPU 315 reads and executes a program stored in the memory 316 to a work area of the memory, and controls each component and the like through the execution of the program by a processor, so that hardware and software cooperate to realize a functional module matching a predetermined purpose.

### Configuration of Guiding Device

Next, a configuration of the guiding device 4 will be described.

In FIG. 1, the guiding device 4 is inserted into the joint cavity C1 through the second portal P2, and guides the insertion of the distal end portion of the ultrasound cutting unit 312 in the treatment instrument 301 into the joint cavity C1.

The guiding device 4 includes a guide main body 401, a handle portion 402, and a drainage unit 403 with a cock.

The guide main body 401 has a cylindrical shape and has a through hole 401a through which the ultrasound cutting unit 312 is inserted (refer to FIG. 1). The guide main body 401 restricts traveling of the ultrasound cutting unit 312 inserted into the through hole 401a in a certain direction and guides movement of the ultrasound cutting unit 312. In the embodiment, a cross-sectional shape orthogonal to the central axis on the outer peripheral surface and the inner peripheral surface of the guide main body 401 is substantially circular. The guide main body 401 is tapered toward the distal end thereof. That is, a distal end surface 401b of the guide main body 401 is an inclined surface that obliquely intersects the central axis.

The drainage unit 403 with the cock is provided on the outer peripheral surface of the guide main body 401 and has a cylindrical shape communicating with the inside of the guide main body 401. One end of a drainage tube 505 of the perfusion device 5 is connected to the drainage unit 403 with the cock, and the guide main body 401 and the drainage tube 505 of the perfusion device 5 communicate with each other. This flow path is configured to be openable and closable by an operation of a cock (not illustrated) provided in the drainage unit 403 with the cock.

### Configuration of Perfusion Device

Next, a configuration of the perfusion device 5 will be described.

In FIG. 1, the perfusion device 5 delivers a perfusate such as sterilized physiological saline into the joint cavity C1 and discharges the perfusate to the outside of the joint cavity C1.

The perfusion device 5 includes a liquid source 501, a liquid feeding tube 502, a liquid feeding pump 503, a drainage bottle 504, the drainage tube 505, and a drainage pump 506 (refer to FIG. 1).

The liquid source 501 stores a perfusate therein. The liquid feeding tube 502 is connected to the liquid source 501. The perfusate is sterilized physiological saline or the like. The liquid source 501 is configured using, for example, a bottle or the like.

The liquid feeding tube 502 has one end connected to the liquid source 501 and the other end connected to the endoscope 201.

The liquid feeding pump 503 feeds the perfusate from the liquid source 501 toward the endoscope 201 through the liquid feeding tube 502. The perfusate delivered to the endoscope 201 is delivered into the joint cavity C1 from a liquid delivery hole formed in the distal end portion of the insertion unit 211.

The drainage bottle 504 stores the perfusate discharged to the outside of the joint cavity C1. The drainage tube 505 is connected to the drainage bottle 504.

The drainage tube 505 has one end connected to the guiding device 4 and the other end connected to the drainage bottle 504.

The drainage pump 506 discharges the perfusate in the joint cavity C1 to the drainage bottle 504 through a flow path of the drainage tube 505 from the guiding device 4 inserted into the joint cavity C1. Note that, in a first embodiment, a description will be given using the drainage pump 506, but the present disclosure is not limited thereto, and a suction device provided in a facility may be used.

### Configuration of Illumination Device

Next, a configuration of the illumination device 6 will be described.

In FIG. 1, the illumination device 6 includes two light sources that emit two types of illumination light having different wavelength bands. The two types of illumination light are, for example, white light that is visible light and infrared light that is invisible light. The illumination light from the illumination device 6 is propagated to the endoscope 201 via a light guide and is emitted from the distal end of the endoscope 201.

### Functional Configuration of Entire Treatment System

Next, a functional configuration of the entire treatment system will be described.

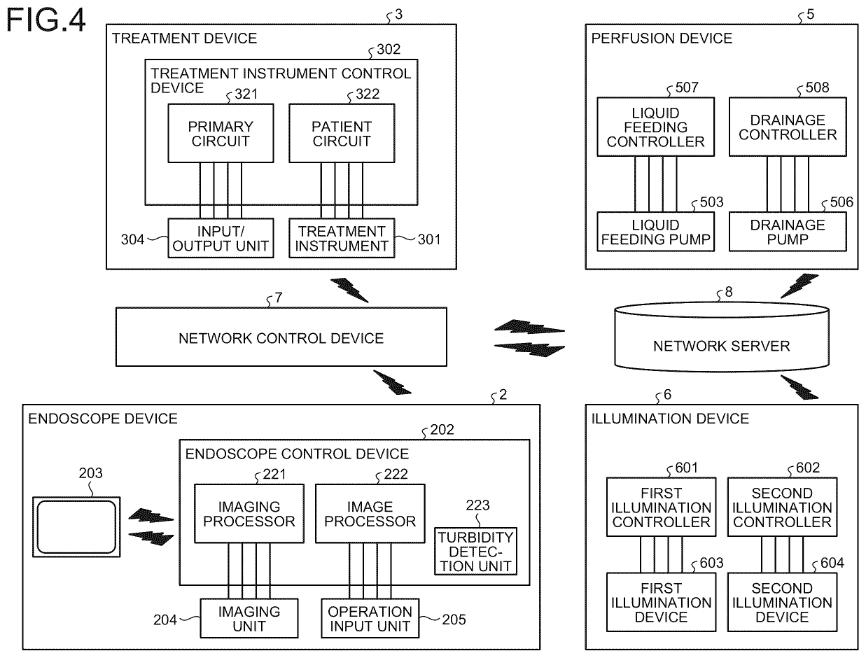

FIG. 4 is a block diagram illustrating an outline of a functional configuration of the entire treatment system 1.

The treatment system 1 illustrated in FIG. 4 further includes a network control device 7 that controls communication of the entire system and a network server 8 that stores various types of data, in addition to the above-described configuration (refer to FIG. 1).

The network control device 7 is communicably connected to the endoscope device 2, the treatment device 3, the perfusion device 5, the illumination device 6, and the network server 8. Although FIG. 4 illustrates a case in which the devices are wirelessly connected to each other, the devices may be connected to each other by wire. Hereinafter, detailed functional configurations of the endoscope device 2, the treatment device 3, the perfusion device 5, and the illumination device 6 will be described.

The network server 8 is communicably connected to the endoscope device 2, the treatment device 3, the perfusion device 5, the illumination device 6, and the network control device 7. The network server 8 stores various types of data of respective devices configuring the treatment system 1. The network server 8 includes, for example, a processor having hardware such as a CPU and a memory such as a hard disk drive (HDD) and a solid state drive (SSD).

### Functional Configuration of Endoscope Device

Next, a functional configuration of the above-described endoscope device 2 will be described.

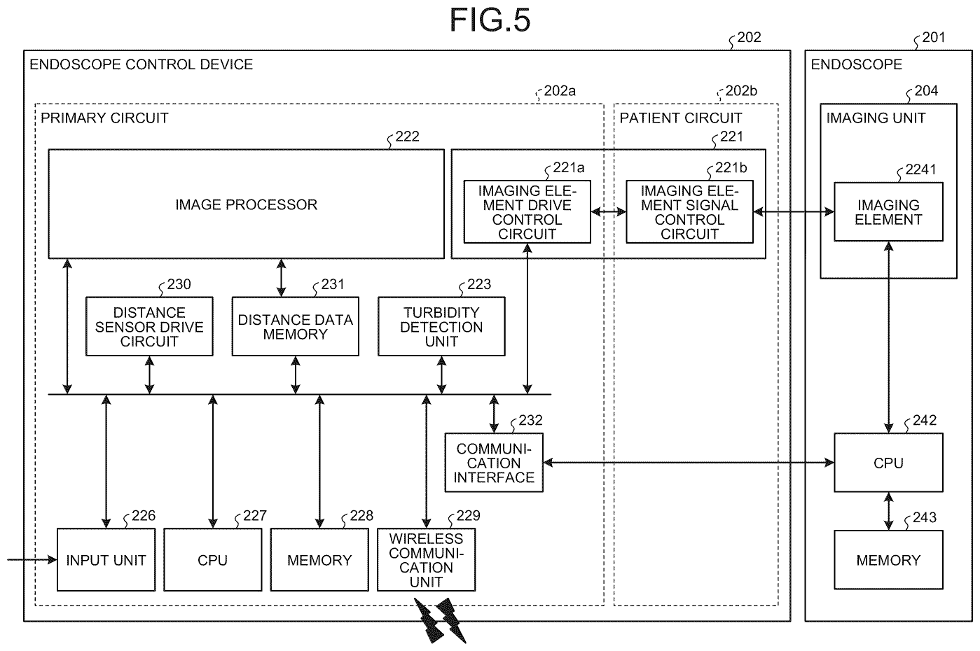

FIG. 5 is a block diagram illustrating a detailed functional configuration of the endoscope device 2.

As illustrated in FIGS. 4 and 5, the endoscope device 2 includes the endoscope control device 202, the display device 203, an imaging portion 204 provided in the endoscope 201, and an operation input unit 205.

The endoscope control device 202 includes an imaging processor 221 (an image acquisition unit), an image processor 222, a turbidity detection unit 223, an input unit 226, a CPU 227, a memory 228, a wireless communication unit 229, a distance sensor drive circuit 230, a distance data memory 231, and a communication interface 232.

The imaging processor 221 includes an imaging element drive control circuit 221a that performs drive control of an imaging element 2241 included in the imaging portion 204 provided in the endoscope 201, and an imaging element signal control circuit 221b that is provided in a patient circuit 202b electrically insulated from a primary circuit 202a and performs signal control of an imaging element 224a. The imaging element drive control circuit 221a is provided in the primary circuit 202a. Furthermore, the imaging element signal control circuit 221b is provided in the patient circuit 202b electrically insulated from the primary circuit 202a.

The image processor 222 performs predetermined image processing on input image data (RAW data) via a bus, and outputs the image data to the display device 203. The image processor 222 is configured using, for example, a processor having hardware of a digital signal processor (DSP) or a field-programmable gate array (FPGA). The image processor 222 reads and executes a program stored in the memory 228 to a work area of the memory, and controls each component and the like through execution of the program by a processor, so that hardware and software cooperate to realize a functional module matching a predetermined purpose. Note that a detailed functional configuration of the image processor 222 will be described later.

The turbidity detection unit 223 detects turbidity of a visual field of the endoscope 201 in the joint cavity C1 based on information on the turbidity of the visual field of the endoscope 201. Here, the information on turbidity is, for example, a value obtained from image data generated by the endoscope 201, a physical property value (turbidity) of a perfusate, impedance acquired from the treatment device 3, and the like.

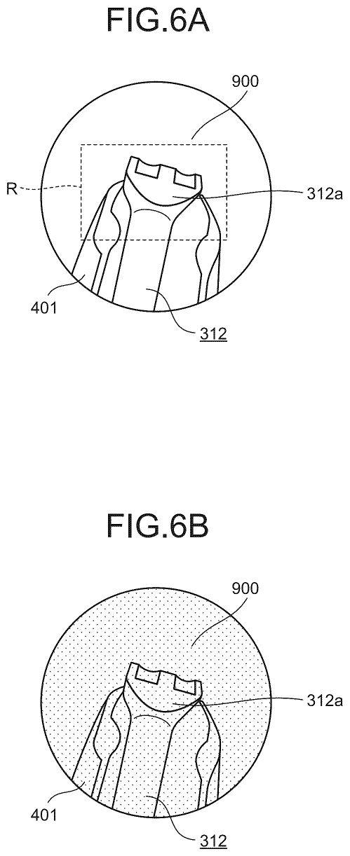

FIG. 6A is a diagram illustrating a state in which the visual field of the endoscope 201 is good.

FIG. 6B is a diagram illustrating a state in which the visual field of the endoscope 201 is poor.

Note that each of FIGS. 6A and 6B is a diagram schematically illustrating a display image corresponding to image data that is the visual field of the endoscope 201 when an operator forms a bone hole in a lateral femoral condyle 900. Here, FIG. 6B schematically illustrates a state in which the visual field of the endoscope 201 becomes turbid due to the bone pulverized into fine particles by drive of the ultrasound cutting unit 312. That is, FIG. 6B is an example of a display image corresponding to image data (turbid image data) captured in a state in which turbidity occurs in the perfusate and the visual field of the endoscope 201 is turbid. In FIG. 6B, fine bones are represented by dots.

In FIG. 5, the input unit 226 receives an input of a signal input by the operation input unit 205 and an input of a signal from each of the devices configuring the treatment system 1.

The CPU 227 integrally controls the operation of the endoscope control device 202. The CPU 227 reads and executes a program stored in the memory 228 to a work area of the memory, and controls each component and the like through execution of the program by a processor, so that hardware and software cooperate to control the operation of each unit of the endoscope control device 202.

The memory 228 stores various types of information necessary for the operation of the endoscope control device 202, various programs executed by the endoscope control device 202, image data captured by the imaging portion 204, and the like. The memory 228 is configured using, for example, a random access memory (RAM), a read only memory (ROM), a frame memory, or the like.

The wireless communication unit 229 is an interface for performing wireless communication with other devices. The wireless communication unit 229 includes, for example, a communication module capable of performing Wi-Fi (registered trademark), Bluetooth (registered trademark), or the like.

The distance sensor drive circuit 230 drives a distance sensor (not illustrated) that measures a distance to a predetermined target object in an image captured by the imaging portion 204. Note that, in the first embodiment, the distance sensor may be provided in the imaging element 2241. Then, the imaging element 2241 may be provided with a phase difference pixel capable of measuring a distance from the imaging element 2241 to a predetermined target object, instead of an effective pixel. Of course, a time of flight (ToF) sensor or the like may be provided in the vicinity of the distal end of the endoscope 201.

The distance data memory 231 stores distance data detected by the distance sensor. The distance data memory 231 includes, for example, a RAM, a ROM, and the like.

The communication interface 232 is an interface for communicating with the imaging portion 204.

In the above-described configuration, components other than the imaging element signal control circuit 221b are provided in the primary circuit 202a, and are connected to each other by a bus wiring.

The imaging portion 204 is provided in the endoscope 201. The imaging portion 204 includes the imaging element 2241, a CPU 242, and a memory 243.

According to the control of the CPU 242, the imaging element 2241 generates image data by capturing a subject image formed by one or a plurality of optical systems (not illustrated), and outputs the generated image data to the endoscope control device 202. The imaging element 2241 is configured using a charge coupled device (CCD) or a complementary metal oxide semiconductor (CMOS) image sensor.

The CPU 242 integrally controls the operation of the imaging portion 204. The CPU 242 reads and executes a program stored in the memory 243 to a work area of the memory, and controls each component and the like through execution of the program by a processor, so that hardware and software cooperate to control the operation of the imaging portion 204.

The memory 243 stores various types of information necessary for the operation of the imaging portion 204, various programs executed by the endoscope 201, image data generated by the imaging portion 204, and the like. The memory 243 includes a RAM, a ROM, a frame memory, and the like.

The operation input unit 205 is configured using an input interface such as a mouse, a keyboard, a touch panel, or a microphone, and receives an operation input of the endoscope device 2 by an operator.

### Functional Configuration of Treatment Device

Next, a functional configuration of the treatment device 3 will be described.

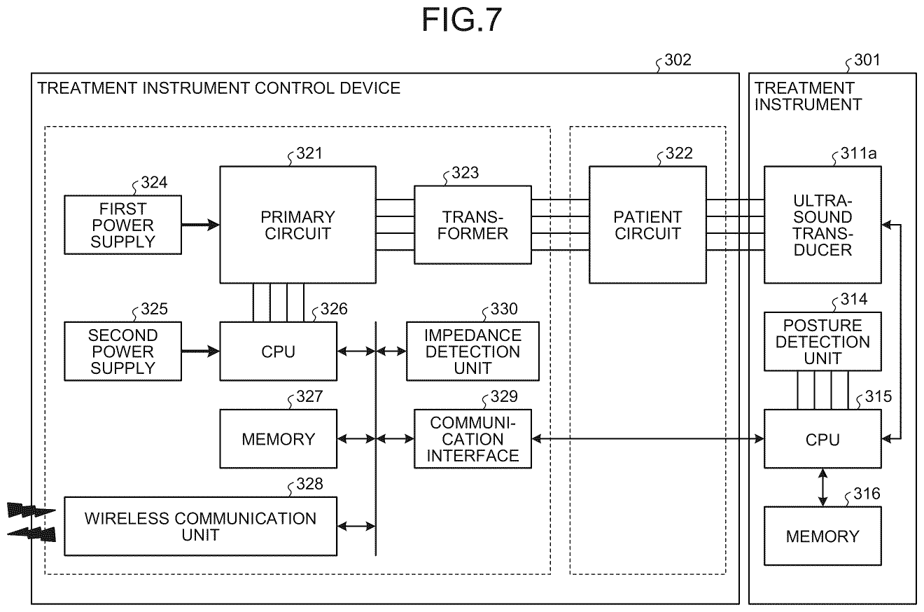

FIG. 7 is a block diagram illustrating a detailed functional configuration of the treatment device 3.

As illustrated in FIGS. 4 and 7, the treatment device 3 includes the treatment instrument 301, the treatment instrument control device 302, and an input/output unit 304.

The treatment instrument 301 includes the ultrasound transducer 312a, the posture detection unit 314, the CPU 315, and the memory 316.

The posture detection unit 314 detects a posture of the treatment instrument 301 and outputs a detection result to the CPU 315. The posture detection unit 314 includes at least one of an acceleration sensor and an angular velocity sensor.

The CPU 315 integrally controls the operation of the treatment instrument 301 including the ultrasound transducer 312a. The CPU 315 reads and executes the program stored in the memory 316 to the work area of the memory, and controls each component and the like through the execution of the program by the processor, so that the hardware and the software cooperate to realize a functional module matching a predetermined purpose.

The memory 316 stores various types of information necessary for the operation of the treatment instrument 301, various programs executed by the treatment instrument 301, and identification information for identifying the type, the manufacturing date, the performance, and the like of the treatment instrument 301.

The treatment instrument control device 302 includes a primary circuit 321, a patient circuit 322, a transformer 323, a first power supply 324, a second power supply 325, a CPU 326, a memory 327, a wireless communication unit 328, a communication interface 329, and an impedance detection unit 330.

The primary circuit 321 generates power to be supplied to the treatment instrument 301. The patient circuit 322 is electrically insulated from the primary circuit 321. The transformer 323 electromagnetically connects the primary circuit 321 and the patient circuit 322 to each other. The first power supply 324 is a high-voltage power supply that supplies drive power for the treatment instrument 301. The second power supply 325 is a low-voltage power supply that supplies drive power for a control circuit in the treatment instrument control device 302.

The CPU 326 integrally controls the operation of the treatment instrument control device 302. The CPU 326 reads and executes a program stored in the memory 327 to a work area of the memory, and controls each component and the like through execution of the program by a processor, so that hardware and software cooperate to control the operation of each unit of the treatment instrument control device 302.

The memory 327 stores various types of information necessary for the operation of the treatment instrument control device 302, various programs executed by the treatment instrument control device 302, and the like. The memory 327 includes a RAM, a ROM, and the like.

The wireless communication unit 328 is an interface for performing wireless communication with other devices. The wireless communication unit 328 includes, for example, a communication module capable of performing Wi-Fi (registered trademark), Bluetooth (registered trademark), and the like.

The communication interface 329 is an interface for performing communication with the treatment instrument 301.

The impedance detection unit 330 detects impedance when the treatment instrument 301 is driven, and outputs a detection result to the CPU 326. Specifically, the impedance detection unit 330 is electrically connected between the first power supply 324 and the primary circuit 321, for example, detects the impedance of the treatment instrument 301 based on voltage and current supplied by the first power supply 324, and outputs the detection result to the CPU 326. This impedance changes depending on a degree of turbidity (cloudiness) of a perfusate, in which the turbidity is generated by bone meal generated by treatment by the treatment instrument 301. That is, the impedance detection unit 330 detects turbidity of the perfusate.

The input/output unit 304 is configured using an input interface such as a mouse, a keyboard, a touch panel, and a microphone and an output interface such as a monitor and a speaker, and outputs an operation input of the endoscope device 2 by an operator and various types of information to be notified to the operator (refer to FIG. 4).

### Functional Configuration of Perfusion Device

Next, a functional configuration of the perfusion device 5 will be described.

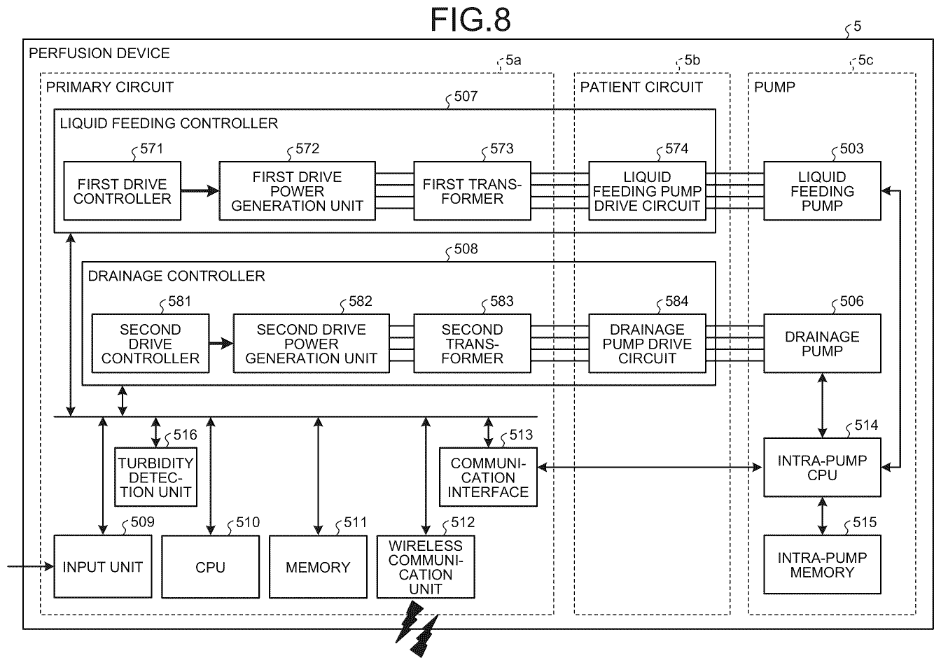

FIG. 8 is a block diagram illustrating a detailed functional configuration of the perfusion device 5.

As illustrated in FIGS. 4 and 8, the perfusion device 5 includes the liquid feeding pump 503, the drainage pump 506, a liquid feeding controller 507, a drainage controller 508, an input unit 509, a CPU 510, a memory 511, a wireless communication unit 512, a communication interface 513, an intra-pump CPU 514, an intra-pump memory 515, and a turbidity detection unit 516.

The liquid feeding controller 507 includes a first drive controller 571, a first drive power generation unit 572, a first transformer 573, and a liquid feeding pump drive circuit 574.

The first drive controller 571 controls drive of the first drive power generation unit 572 and the liquid feeding pump drive circuit 574.

The first drive power generation unit 572 generates drive power of the liquid feeding pump 503 and supplies the drive power to the first transformer 573.

The first transformer 573 electromagnetically connects the first drive power generation unit 572 to the liquid feeding pump drive circuit 574.

In the liquid feeding controller 507 configured as such, the first drive controller 571, the first drive power generation unit 572, and the first transformer 573 are provided in a primary circuit 5a. The liquid feeding pump drive circuit 574 is provided in a patient circuit 5b electrically insulated from the primary circuit 5a.

The drainage controller 508 includes a second drive controller 581, a second drive power generation unit 582, a second transformer 583, and a drainage pump drive circuit 584.

The second drive controller 581 controls drive of the second drive power generation unit 582 and the drainage pump drive circuit 584.

The second drive power generation unit 582 generates drive power for the drainage pump 506 and supplies the generated drive power to the second transformer 583.

The second transformer 583 electromagnetically connects the second drive power generation unit 582 to the drainage pump drive circuit 584.

In the drainage controller 508 configured as such, the second drive controller 581, the second drive power generation unit 582, and the second transformer 583 are provided in the primary circuit 5a. The drainage pump drive circuit 584 is provided in the patient circuit 5b electrically insulated from the primary circuit 5a.

The input unit 509 receives an operation input (not illustrated) or an input of a signal from each of the devices configuring the treatment system 1, and outputs the received signal to the CPU 510 and the intra-pump CPU 514.

The CPU 510 and the intra-pump CPU 514 cooperate to integrally control the operation of the perfusion device 5. The CPU 510 reads and executes a program stored in the memory 511 to a work area of the memory, and controls each component and the like through execution of the program by a processor, so that hardware and software cooperate to control the operation of each unit of the perfusion device 5.

The memory 511 stores various types of information necessary for the operation of the perfusion device 5 and various programs executed by the perfusion device 5. The memory 511 includes a R-AM, a ROM, and the like.

The wireless communication unit 512 is an interface for performing wireless communication with other devices. The wireless communication unit 512 is configured using, for example, a communication module capable of performing Wi-Fi, Bluetooth, or the like.

The communication interface 513 is an interface for performing communication with the liquid feeding pump 503 and the endoscope 201.

The intra-pump memory 515 stores various types of information necessary for the operation of the liquid feeding pump 503 and the drainage pump 506 and various programs executed by the liquid feeding pump 503 and the drainage pump 506.

The turbidity detection unit 516 detects turbidity of a perfusate based on any one or more of a physical property value, absorbance, impedance, and a resistance value of the perfusate flowing in the drainage tube 505, and outputs a detection result to the CPU 510.

In the perfusion device 5 configured as such, the input unit 509, the CPU 510, the memory 511, the wireless communication unit 512, the communication interface 513, and the turbidity detection unit 516 are provided in the primary circuit 5a. Further, the intra-pump CPU 514 and the intra-pump memory 515 are provided in a pump Sc. Note that the intra-pump CPU 514 and the intra-pump memory 515 may be provided around the liquid feeding pump 503 or may be provided around the drainage pump 506.

Functional Configuration of Illumination Device Next, a functional configuration of the illumination device 6 will be described.

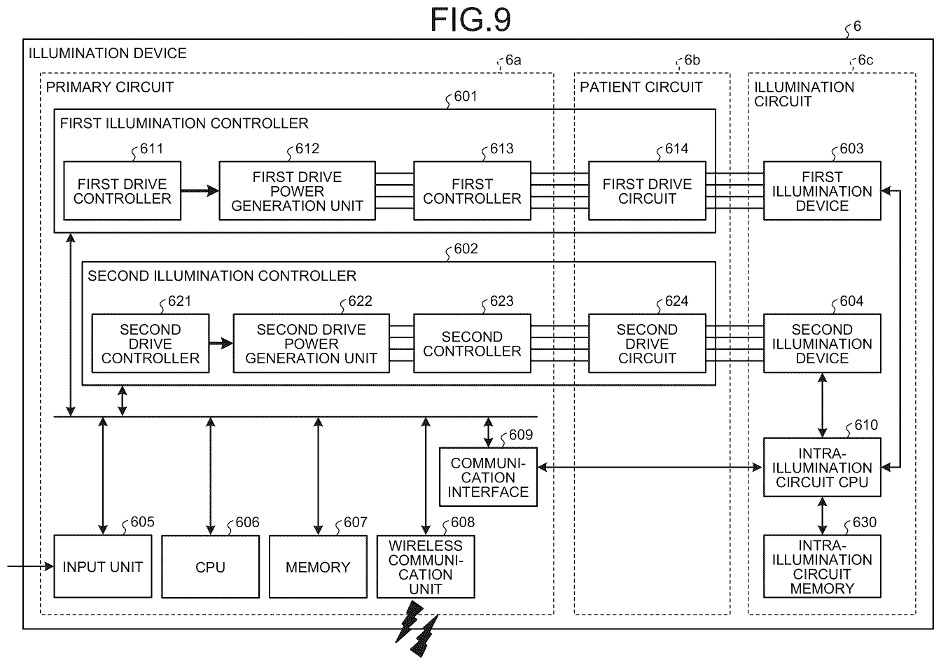

FIG. 9 is a block diagram illustrating a detailed functional configuration of the illumination device 6.

As illustrated in FIGS. 4 and 9, the illumination device 6 includes a first illumination controller 601, a second illumination controller 602, a first illumination device 603, a second illumination device 604, an input unit 605, a CPU 606, a memory 607, a wireless communication unit 608, a communication interface 609, an intra-illumination circuit CPU 610, and an intra-illumination circuit memory 630.

The first illumination controller 601 includes a first drive controller 611, a first drive power generation unit 612, a first controller 613, and a first drive circuit 614.

The first drive controller 611 controls drive of the first drive power generation unit 612, the first controller 613, and the first drive circuit 614.

According to the control of the first drive controller 611, the first drive power generation unit 612 generates drive power for the first illumination device 603, and outputs the drive power to the first controller 613.

The first controller 613 controls a light output of the first illumination device 603 by controlling the first drive circuit 614 according to the drive power input from the first drive power generation unit 612.

Under the control of the first controller 613, the first drive circuit 614 drives the first illumination device 603 to output illumination light.

In the first illumination controller 601 configured as such, the first drive controller 611, the first drive power generation unit 612, and the first controller 613 are provided in a primary circuit 6a. The first drive circuit 614 is provided in a patient circuit 6b electrically insulated from the primary circuit 6a.

The second illumination controller 602 includes a second drive controller 621, a second drive power generation unit 622, a second controller 623, and a second drive circuit 624.

The second drive controller 621 controls drive of the second drive power generation unit 622, the second controller 623, and the second drive circuit 624.

According to the control of the second drive controller 621, the second drive power generation unit 622 generates drive power for the second illumination device 604, and outputs the drive power to the second controller 623.

The second controller 623 controls a light output of the second illumination device 604 by controlling the second drive circuit 624 according to the drive power input from the second drive power generation unit 622.

According to the control of the second controller 623, the second drive circuit 624 drives the second illumination device 604 to output illumination light.

In the second illumination controller 602 configured as such, the second drive controller 621, the second drive power generation unit 622, and the second controller 623 are provided in the primary circuit 6a. The second drive circuit 624 is provided in the patient circuit 6b electrically insulated from the primary circuit 6a.

The first illumination device 603 sequentially irradiates a subject with light in a wavelength band of visible light (hereinafter, simply referred to as “visible light”) and different light in a wavelength band outside the visible light (hereinafter, simply referred to as “invisible light”) as first illumination light for irradiation of the subject via the endoscope 201. Here, the visible light is at least one of light in a blue wavelength band (400 nm to 500 nm), light in a green wavelength band (480 nm to 600 nm), and light in a red wavelength band (570 nm to 680 nm). The invisible light is infrared light (800 nm to 2500 nm). Note that a configuration of the first illumination device 603 will be described later.

The second illumination device 604 is configured to irradiate the subject with special light as second illumination light for irradiation of the subject via the endoscope 201, and may be used as illumination for detecting subject information. Alternatively, the first illumination device 603 may be light in a wavelength band of visible light, and the second illumination device 604 may be illumination in a wavelength band of invisible light.

The input unit 605 receives an input of a signal from each of the devices configuring the treatment system 1, and outputs the received signal to the CPU 606 and the intra-illumination circuit CPU 610.

The CPU 606 and the intra-illumination circuit CPU 610 cooperate to integrally control the operation of the illumination device 6. The CPU 606 reads and executes a program stored in the memory 607 to a work area of the memory, and controls each component and the like through execution of the program by a processor, so that hardware and software cooperate to control the operation of each unit of the illumination device 6.

The memory 607 stores various types of information necessary for the operation of the illumination device 6 and various programs executed by the illumination device 6. The memory 607 includes a RAM, a ROM, and the like.

The wireless communication unit 608 is an interface for performing wireless communication with other devices. The wireless communication unit 608 is configured using, for example, a communication module capable of performing Wi-Fi, Bluetooth, or the like.

The communication interface 609 is an interface for communicating with an illumination circuit 6c.

The intra-illumination circuit memory 630 stores various types of information and programs necessary for the operation of the first illumination device 603 and the second illumination device 604. The intra-illumination circuit memory 630 includes a RAM, a ROM, and the like.

In the illumination device 6 configured as such, the input unit 605, the CPU 606, the memory 607, the wireless communication unit 608, and the communication interface 609 are provided in the primary circuit 6a. In addition, the first illumination device 603, the second illumination device 604, the intra-illumination circuit CPU 610, and the intra-illumination circuit memory 630 are provided in the illumination circuit 6c.

### Configuration of First Illumination Device

Next, a configuration of the above-described first illumination device will be described.

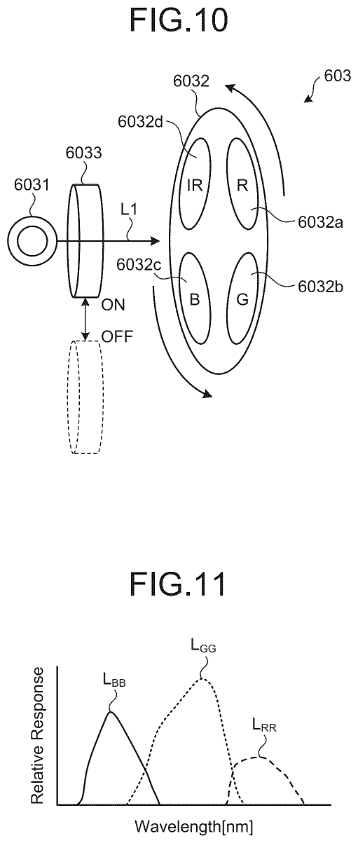

FIG. 10 is a schematic diagram illustrating a schematic configuration of the first illumination device 603.

The first illumination device 603 illustrated in FIG. 10 includes a light source 6031 capable of emitting illumination light, a rotary filter 6032, and an IR transmission filter 6033 disposed on an optical path L1 of the illumination light emitted by the light source 6031 to be movable forward and rearward by a drive unit (not illustrated).

The light source 6031 is configured using a light source such as a halogen lamp.

The light source 6031 emits light according to the drive of the first drive circuit 614.

The rotary filter 6032 includes a red filter 6032a that transmits light in a red wavelength band (570 nm to 680 nm), a green filter 6032b that transmits light in a green wavelength band (480 nm to 600 nm), a blue filter 6032c that transmits light in a blue wavelength band (400 nm to 500 nm), and a transparent filter 6032d that transmits light transmitted through the IR transmission filter 6033 (870 nm to 1080 nm). The rotary filter 6032 is rotated by a drive unit (not illustrated) so that any one of the red filter 6032a, the green filter 6032b, the blue filter 6032c, and the transparent filter 6032d is disposed on the optical path of white light emitted by the light source 6031.

The IR transmission filter 6033 is disposed on the optical path L1 of the illumination light emitted by the light source 6031 to be movable forward and rearward by the drive unit (not illustrated). The IR transmission filter 6033 transmits infrared light (870 nm to 1080 nm) that is a wavelength band of invisible light included in the illumination light emitted by the light source 6031.

### Transmission Characteristic of Each Filter

Next, transmission characteristic of each filter will be described.

FIG. 11 is a diagram illustrating a relationship between transmission characteristics and wavelength bands of the red filter 6032a, the green filter 6032b, and the blue filter 6032c.

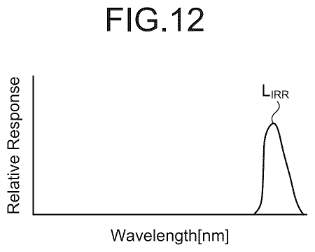

FIG. 12 is a diagram illustrating a relationship between a transmission characteristic and a wavelength band of the IR transmission filter 6033.

In FIGS. 11 and 12, a horizontal axis represents a wavelength, and a vertical axis represents transmittance.

In FIG. 11, a curved line L_{RR} indicates the transmission characteristic of the red filter 6032a, a curved line L_{GG} indicates the transmission characteristic of the green filter 6032b, and a curved line L_{BB} indicates the transmission characteristic of the blue filter 6032c. Further, in FIG. 12, a curved line L_{IRR} indicates the transmission characteristic of the IR transmission filter 6033.

As illustrated in FIGS. 11 and 12, the rotary filter 6032 rotates according to the drive of the drive unit (not illustrated) to transmit the light in the red wavelength band, the light in the green wavelength band, the light in the blue wavelength band, and the light in the infrared wavelength band toward a subject.

### Detailed Functional Configuration of Image Processor

Next, a detailed functional configuration of the above-described image processor 222 will be described.

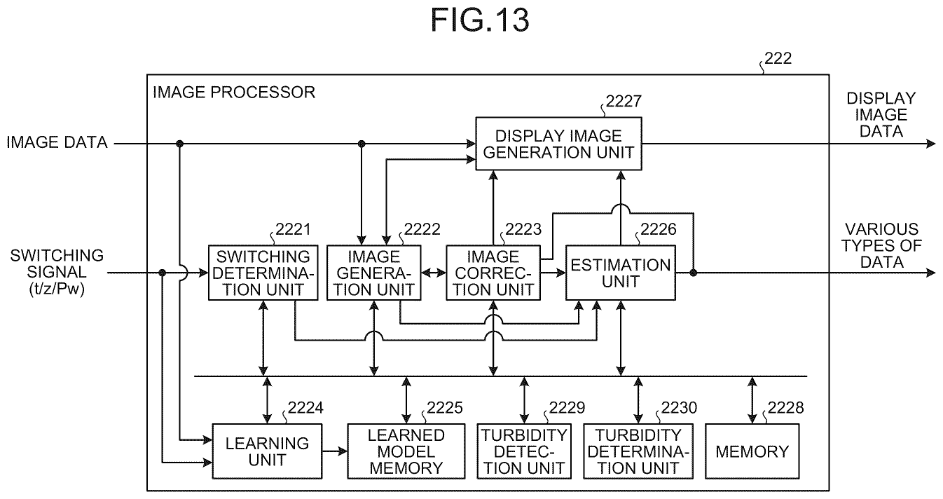

FIG. 13 is a block diagram illustrating a detailed functional configuration of the image processor 222.

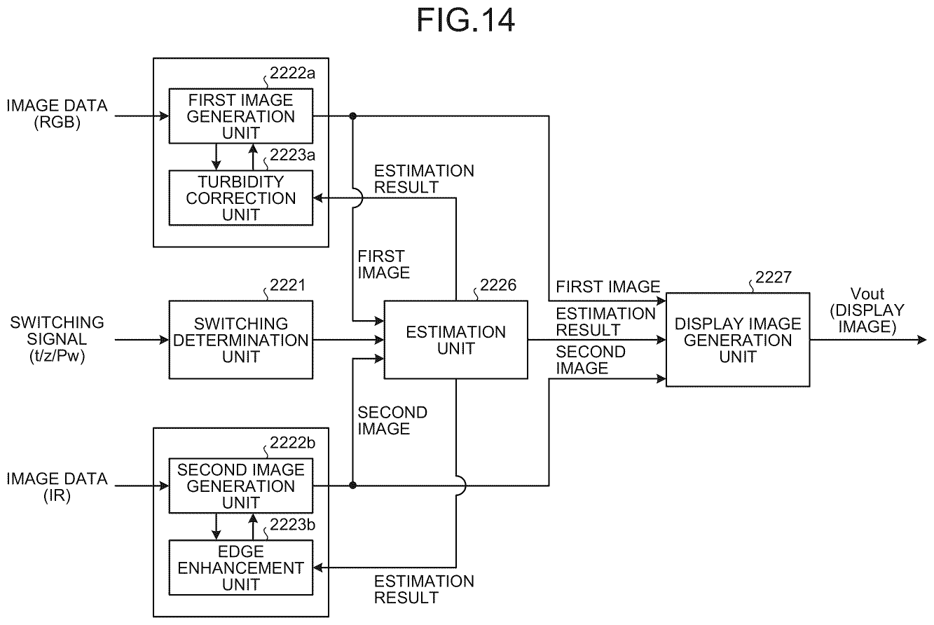

FIG. 14 is a block diagram schematically illustrating exchange of a part of signals configuring the image processor 222.

The image processor 222 illustrated in FIGS. 13 and 14 includes a switching determination unit 2221, an image generation unit 2222, an image correction unit 2223, a learning unit 2224, a learned model memory 2225, an estimation unit 2226, a display image generation unit 2227, a memory 2228, a turbidity detection unit 2229, and a turbidity determination unit 2230.

The switching determination unit 2221 determines a learned model when the estimation unit 2226 to be described later performs estimation on an image corresponding to image data based on one or more switching signals of treatment time t for a living body by the treatment instrument 301 input from the outside, impedance Z that is an electrical characteristic for the living body by the treatment instrument 301 detected by the impedance detection unit 330, and supply power Pw supplied to the treatment instrument 301, and outputs a determination result to the estimation unit 2226. In addition, the switching determination unit 2221 outputs the determination result to the learning unit 2224 via a bus.

The image generation unit 2222 performs predetermined image processing on image data (RAW data) input from the outside to generate a first image corresponding to colored (RGB) first image data or a second image corresponding to second image data that is infrared image data. As illustrated in FIG. 14, the image generation unit 2222 includes a first image generation unit 2222a and a second image generation unit 2222b. Note that, in the embodiment, the image generation unit 2222 functions as an image acquisition unit that acquires image data.

When the first illumination device 603 sequentially emits light in each of the red, green, and blue wavelength bands, the first image generation unit 2222a performs predetermined image processing on three pieces of image data of red, green, and blue generated by the endoscope 201, thereby generating a first image. Here, examples of the predetermined image processing include synthesis processing of mixing the three image data of red, green, and blue at a predetermined ratio to generate a white image, color correction processing, black level correction processing, noise reduction processing, y correction processing, and the like.

When the first illumination device 603 sequentially emits infrared light, the second image generation unit 2222b performs predetermined image processing on the second image data generated by the endoscope 201 to generate a second image. Here, the predetermined image processing includes color correction processing, black level correction processing, noise reduction processing, y correction processing, and the like.

The image correction unit 2223 performs image correction on the first image and the second image generated by the image generation unit 2222, and outputs the corrected images to the display image generation unit 2227 or the learning unit 2224. The image correction unit 2223 includes a turbidity correction unit 2223a and an edge enhancement unit 2223b.

The turbidity correction unit 2223a generates first correction image data obtained by performing gradation correction on the first image generated by the first image generation unit 2222a, and outputs a first correction image corresponding to the first correction image data (hereinafter, simply referred to as a “first correction image”) to the display image generation unit 2227 or the learning unit 2224. Specifically, the turbidity correction unit 2223a generates the first correction image by performing the gradation correction on the first image to remove deterioration factors of visibility due to turbidity (turbidity component) included in the first image. Note that details of the turbidity correction unit 2223a will be described later.

When contrast of the image generated by the second image generation unit 2222b is low and sufficient contrast is not obtained, the edge enhancement unit 2223b performs known edge enhancement processing on the second image to generate second correction image data, and outputs a second correction image corresponding to the second correction image data (hereinafter, simply referred to as a “second correction image”) to the display image generation unit 2227 or the learning unit 2224.

The learning unit 2224 is provided to perform learning using teacher data in advance before treatment. The learning unit 2224 is configured to perform learning when treatment is not performed, for example, when learning is performed in advance. Therefore, in general, a description will be given on the assumption that learning in the learning unit 2224 is not performed when treatment is performed, and learning in the learning unit 2224 is performed when treatment is not performed. The learning unit 2224 generates a learned model in advance by performing machine learning using teacher data (learning data set or training data) including a plurality of pieces of image data (PAW data) generated by the endoscope 201, the first image, the second image, the first correction image, the second correction image, the treatment time t to a living body by the treatment instrument 301 input from the outside, the impedance Z detected by the impedance detection unit 330, the supply power Pw supplied to the treatment instrument 301, and the like. Specifically, the learning unit 2224 generates a learned model in advance by performing machine learning using teacher data in which a plurality of pieces of treatment image data of image data (PAW) generated by the endoscope 201, the first image, the second image, the first correction image in which turbidity is reduced or removed by the turbidity correction unit 2223a, and the second correction image in which edge enhancement is performed by the edge enhancement unit 2223b, and a plurality of pieces of annotation image data to which an annotation of a target object included in the plurality of second images, the first correction image, and the second correction image is applied are defined as input data, and an identification result of identifying the target object included in the first image is defined as output data. The learning unit 2224 generates a learned model in advance using a known machine learning method. Examples of the machine learning include deep learning using a neural network, but machine learning based on other methods may be applied. Examples of a statistical model of machine learning include a simple linear regression model, Ridge regression, Lasso regression, Elastic Net regression, random forest regression, rule fit regression, gradient boosting tree, extra tree, support vector regression, Gaussian process regression, k-nearest neighbor regression, and kernel ridge regression.

Furthermore, the learning unit 2224 may generate a learned model of each of the treatment time t to the living body by the treatment instrument 301 input from the outside, the impedance Z detected by the impedance detection unit 330, and the supply power Pw supplied to the treatment instrument 301 by further using teacher data including the treatment time t, the impedance Z, the supply power Pw, and the like as input parameters, and may store the learned model in the learned model memory 2225.

In addition, the learning unit 2224 may generate a learned model by further using teacher data including turbidity (turbidity component) of the first image detected by the turbidity detection unit 2229 as input parameters, and may store the learned model in the learned model memory 2225. Furthermore, the learning unit 2224 may perform relearning by inputting image data input to the image processor 222 as input data to the learned model stored in the learned model memory 2225.

The learned model memory 2225 stores a plurality of learned models. Specifically, the learned model memory 2225 stores learned models respectively corresponding to the treatment time t, the impedance Z, and the supply power Pw. The learned model memory 2225 includes a RAM, a ROM, and the like.

The estimation unit 2226 reads the learned model corresponding to the switching signal input from the switching determination unit 2221 from the learned model memory 2225, estimates the target object included in the first image based on the read learned model and at least one of the first image and the second image, and outputs an estimation result to the display image generation unit 2227. Specifically, the estimation unit 2226 uses the switching signal, the first image, and the second image as input parameters, and outputs the target object included in the first image as output parameters to the display image generation unit 2227. Here, the target object is the treatment instrument 301 in the liquid in which a powdery material is diffused, a powdery material diffused in the liquid, a position of the powdery material, a position of the treatment instrument 301 in the first image, a position of an index portion provided in the treatment instrument 301, a movement amount of the index portion of the treatment instrument 301, treatment waste generated by treatment by the treatment instrument 301, a shape of the treatment instrument 301, and the like.

The display image generation unit 2227 generates display image data based on at least one of the first image and the second image and the target object estimated by the estimation unit 2226, converts a display image corresponding to the display image data (hereinafter, simply referred to as a “display image”) into a predetermined format system, for example, converts an RGB system into a YCbCr system, and outputs the display image data to the display device 203. Specifically, the display image generation unit 2227 generates the display image obtained by superimposing position information regarding the position of an area of the target object estimated by the estimation unit 2226 on the first image.

The memory 2228 stores various types of information necessary for the operation of the image processor 222, various programs executed by the image processor 222, various types of image data, and the like. The memory 2228 includes a RAM, a ROM, a frame memory, and the like.

The turbidity detection unit 2229 detects a change in gradation from at least a partial area of the first image based on the first image generated by the image generation unit 2222, and outputs a detection result to the turbidity determination unit 2230 and the learning unit 2224. Specifically, the turbidity detection unit 2229 detects the turbidity of the visual field in the endoscope 201 as at least a partial area of the first image based on the first image generated by the image generation unit 2222. In a method of detecting turbidity by the turbidity detection unit 2229, the turbidity is detected by the same method of the turbidity component of the turbidity estimation unit 2226a of the image correction unit 2223 to be described later, and thus a detailed detection method is omitted.

The turbidity determination unit 2230 determines whether the turbidity detected by the turbidity detection unit 2229 is equal to or greater than a predetermined value, and outputs a determination result to the display image generation unit 2227. Here, the predetermined value is, for example, a value of a level at which an operation spot in the visual field of the endoscope 201 disappears due to turbidity. For example, the value of the level at which the operation spot disappears is a value of high luminance and low chroma (high luminance white).

### Detailed Functional Configuration of Turbidity Correction Unit

Next, a detailed functional configuration of the turbidity correction unit 2223a will be described.

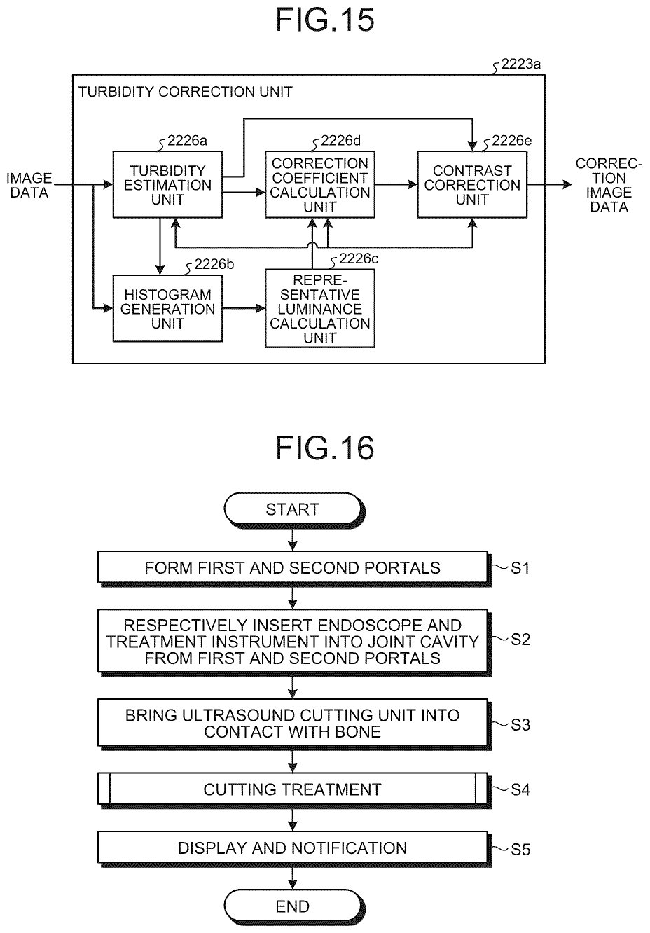

FIG. 15 is a block diagram illustrating a detailed functional configuration of the turbidity correction unit 2223a.

The turbidity correction unit 2223a illustrated in FIG. 15 includes a turbidity estimation unit 2226a, a histogram generation unit 2226b, a representative luminance calculation unit 2226c, a correction coefficient calculation unit 2226d, and a contrast correction unit 2226e.

The turbidity estimation unit 2226a estimates a turbidity component in each pixel in the first image. Here, the turbidity component in each pixel is a degree of turbidity of bone meal and debris dissolved in a perfusate as a factor that causes deterioration in gradation in the first image. As a factor of causing deterioration in image quality, in addition to a phenomenon caused by dissolution of a perfusate of a biological tissue such as bone meal, debris, blood, and bone marrow, a phenomenon of smoke or sparks during treatment of the treatment instrument 301 can also be mentioned. Hereinafter, the turbidity when the bone meal is dissolved in the perfusate and the perfusate becomes cloudy will be described. The perfusate in which the biological tissue is dissolved has characteristics of high brightness, low chroma (low color reproduction), and low contrast.

Therefore, the turbidity estimation unit 2226a estimates a turbidity component of the visual field of the endoscope 201 by calculating contrast, or luminance and saturation of the first image. Specifically, the turbidity estimation unit 2226a estimates a turbidity component H(x, y) based on an R value, a G value, and a B value of a pixel in coordinates (x, y) in the first image.

Here, when the R value, the G value, and the B value in coordinates (x, y) are denoted by Ir, Ig, and Ib, respectively, the turbidity component H(x, y) of the pixel in coordinates (x, y) is estimated by the following Formula (1).

$$H ( x , y ) = min ( Ir , Ig , Ib ) \qquad ( 1 )$$

The turbidity estimation unit 2226a performs the above-described calculation of Formula (1) for each pixel of the first image. The turbidity estimation unit 2226a sets a scan area F (small area) having a predetermined size for the first image. The size of the scan area F is, for example, a predetermined size of m×n (m and n is a natural number) pixels. Hereinafter, a pixel at the center of the scan area F will be described as a reference pixel. Furthermore, in the following description, each pixel around the reference pixel in the scan area F will be described as a neighboring pixel. Furthermore, in the following description, the scan area F is formed in a size of, for example, 5×5 pixels. Of course, the scan area F can be applied even to one pixel.

The turbidity estimation unit 2226a calculates (Ir, Ig, Ib) of each pixel in the scan area F while shifting the position of the scan area F with respect to the first image, and estimates the minimum value as the turbidity component H(x, y) of the reference pixel. In a pixel value of a high luminance and low chroma area in the first image, since the R value, the G value, and the B value are equal and large, the value of min(Ir, Ig, Ib) increases. That is, in the high luminance and low chroma area, the turbidity component H(x, y) has a large value.

On the other hand, in the pixel value in a low luminance or high chroma area, since any one of the R value, the G value, and the B value decreases, the value of min(Ir, Ig, Ib) decreases. That is, in the low luminance or high chroma area, the turbidity component H(x, y) has a small value.

As such, the turbidity component H(x, y) has a larger value as concentration of the bone meal dissolved in the perfusate is higher (as the white color of the bone meal is thicker), and has a smaller value as concentration of the bone meal dissolved in the perfusate is lower. In other words, the turbidity component H(x, y) has a larger value as the color (white) of the perfusate is thicker due to the bone meal dissolved in the perfusate, and has a smaller value as the color of the perfusate is softer.

Note that, although the turbidity estimation unit 2226a estimates the turbidity component H(x, y) by the above-described Formula (1), the disclosure is not limited thereto, and any index indicating high luminance and low chroma can be used as the turbidity component. The turbidity estimation unit 2226a may estimate the turbidity component using any one or more of a local contrast value, an edge intensity, a color density, and a subject distance. In addition, the turbidity detection unit 2229 described above detects turbidity (turbidity component) by a method similar to that of the turbidity estimation unit 2226a.

Based on the turbidity component H(x, y) input from the turbidity estimation unit 2226a, the histogram generation unit 2226b determines a distribution of a histogram in a local area including a reference pixel of the first image and a neighboring pixel around the reference pixel. A degree of change in the turbidity component H(x, y) serves as an index for determining an area to which each pixel belongs in the local area. Specifically, the degree of change in the turbidity component H(x, y) is determined based on a difference in the turbidity component H(x, y) between the reference pixel and the neighboring pixel in the local area.

That is, based on the first image input from the first image generation unit 2222a and the turbidity component H(x, y) input from the turbidity estimation unit 2226a, the histogram generation unit 2226b generates a luminance histogram for a local area including a neighboring pixel for each reference pixel. A general histogram is generated by regarding a pixel value in a target local area as a luminance value and counting frequency of the pixel value one by one.

On the other hand, the histogram generation unit 2226b according to the first embodiment weights a count value for the pixel value of the neighboring pixel according to the turbidity component H(x, y) between the reference pixel and the neighboring pixel in the local area. The count value for the pixel value of the neighboring pixel is, for example, a value in a range of 0.0 to 1.0. In addition, the count value is set so that the larger the difference in the turbidity component H(x, y) between the reference pixel and the neighboring pixel, the smaller the count value, and the smaller the difference in the turbidity component H(x, y) between the reference pixel and the neighboring pixel, the larger the count value. Furthermore, the local area is formed in a size of, for example, 7×7 pixels.

In a general histogram generation, when a histogram is generated only by luminance, luminance of a pixel of interest and luminance of a neighboring pixel having a large value difference are also counted in the same manner. The local histogram is desirably generated according to an image area to which the pixel of interest belongs.

On the other hand, in the generation of the luminance histogram according to the embodiment, the count value for the pixel value of each pixel in the local area in the first image data is set according to the difference in the turbidity component H(x, y) between the reference pixel and each neighboring pixel in the local area in the first image data of the turbidity component H(x, y). Specifically, the count value is calculated using, for example, a Gaussian function so that the larger the difference in the turbidity component H(x, y) between the reference pixel and the neighboring pixel, the smaller the count value, and the smaller the difference in the turbidity component H(x, y) between the reference pixel and the neighboring pixel, the larger the count value (refer to, for example, JP 6720012 B2 or JP 6559229 B2. Here, a haze component is replaced with the turbidity component).

Note that a method of calculating the count value by the histogram generation unit 2226b is not limited to the Gaussian function, and only needs to be determined so that the larger the difference between the values of the reference pixel and the neighboring pixel, the smaller the count value. For example, the histogram generation unit 2226b may calculate the count value using a lookup table or a table approximated by a polygonal line instead of the Gaussian function.

Furthermore, the histogram generation unit 2226b may compare the difference between the values of the reference pixel and the neighboring pixel with a threshold value, and may reduce (for example, set to 0.0) the count value of the neighboring pixel when the difference is equal to or greater than the threshold value.

Furthermore, the histogram generation unit 2226b may not necessarily use the frequency of the pixel value as the count value. For example, the histogram generation unit 2226b may use each of the R value, the G value, and the B value as a count value. In addition, the histogram generation unit 2226b may use the G value that is a luminance value as a count value.

The representative luminance calculation unit 2226c calculates representative luminance based on the statistical information of the luminance histogram input from the histogram generation unit 2226b. The representative luminance is luminance of a low luminance portion, luminance of a high luminance portion, and luminance of an intermediate luminance portion in an effective luminance range of the luminance histogram. The luminance of the low luminance portion is the minimum luminance in the effective luminance range. The luminance of the high luminance portion is the maximum luminance in the effective luminance range. The luminance of the intermediate luminance portion is centroid luminance. The minimum luminance is a luminance at which the cumulative frequency is 5% of the maximum value in the cumulative histogram generated from the luminance histogram. The maximum luminance is a luminance at which the cumulative frequency is 95% of the maximum value in the cumulative histogram generated from the luminance histogram. The centroid luminance is a luminance at which the cumulative frequency is 50% of the maximum value in the cumulative histogram generated from the luminance histogram.

Note that 5%, 50%, and 95%, that are the percentages of the cumulative frequency corresponding to the minimum luminance, the maximum luminance, and the centroid luminance, can be appropriately changed. Furthermore, although the luminance of the intermediate luminance portion is the centroid luminance in the cumulative histogram, the present disclosure is not limited thereto, and the centroid luminance may not necessarily be calculated from the cumulative frequency. For example, the luminance of the intermediate luminance portion can be applied even as the luminance of the maximum frequency of the luminance histogram.

The correction coefficient calculation unit 2226d calculates a correction coefficient for correcting contrast in the local area based on the turbidity component H(x, y) input from the turbidity estimation unit 2226a and the statistical information input from the representative luminance calculation unit 2226c. Specifically, when contrast correction is performed by histogram expansion, the correction coefficient calculation unit 2226d calculates a coefficient for histogram expansion using the centroid luminance and the maximum luminance of the statistical information.

Here, the histogram expansion is processing of enhancing the contrast by expanding the effective luminance range of the histogram (refer to, for example, JP 6720012 B2 or JP 6559229 B2). Note that the correction coefficient calculation unit 2226d uses the histogram expansion as means to implement contrast correction, but the present disclosure is not limited thereto, and for example, histogram flattening may be applied as means to implement contrast correction. For example, the correction coefficient calculation unit 2226d may apply a method using a cumulative histogram or a table approximating a polygonal line as a method of realizing histogram flattening. This cumulative histogram is obtained by sequentially accumulating frequent values of the luminance histogram.

The contrast correction unit 2226e corrects the contrast of the reference pixel of the first image data based on the turbidity component H(x, y) input from the turbidity estimation unit 2226a and the correction coefficient input from the correction coefficient calculation unit 2226d with respect to the first image input from the first image generation unit 2222a (refer to, for example, JP 6720012 B2 or JP 6559229 B2).

The turbidity correction unit 2223a configured as such estimates the turbidity component H(x, y) based on the first image, calculates the luminance histogram and the representative luminance using the estimation result, calculates the correction coefficient for correcting the contrast in the local area, and performs contrast correction based on the turbidity component H(x, y) and the correction coefficient. As a result, the turbidity correction unit 2223a can generate the first correction image in which the turbidity is removed from the first image.

### Outline of Treatment

Next, an outline of treatment performed by an operator using the treatment system 1 will be described.

FIG. 16 is a flowchart illustrating an outline of treatment performed by the operator using the treatment system 1.

Note that an operator who performs the treatment may be one doctor or two or more doctors and assistants.

As illustrated in FIG. 16, first, an operator forms the first portal P1 and the second portal P2 that respectively allow the inside of the joint cavity C1 of the knee joint J1 and the outside of the skin to communicate with each other (step S1).

Subsequently, the operator inserts the endoscope 201 into the joint cavity C1 from the first portal P1, inserts the guiding device 4 into the joint cavity C1 from the second portal P2, and inserts the treatment instrument 301 into the joint cavity C1 by guiding of the guiding device 4 (step S2). Note that, although a case in which the endoscope 201 and the treatment instrument 301 are inserted into the joint cavity C1 from the first portal P1 and the second portal P2 after forming the two portals is described here, the guiding device 4 and the treatment instrument 301 may be inserted into the joint cavity C1 by forming the second portal P2 after forming the first portal P1 and inserting the endoscope 201 into the joint cavity C1.

Thereafter, the operator brings the ultrasound cutting unit 312 into contact with the bone to be treated while visually confirming an endoscopic image in the joint cavity C1 displayed by the display device 203 (step S3).

Subsequently, the operator performs cutting treatment using the treatment instrument 301 while viewing the endoscopic image displayed on the display device 203 (step S4). Note that details of processing of the treatment system 1 in the cutting treatment will be described later.

Thereafter, the display device 203 performs display/notification processing of displaying the inside of the joint cavity C1 and notifying information on the state after the cutting treatment (step S5). For example, the endoscope control device 202 stops the display/notification after a predetermined time elapses after the display/notification processing. The operator ends the treatment using the treatment system 1.

Details of Cutting Treatment Next, details of the cutting treatment in step S4 of FIG. 16 described above will be described.

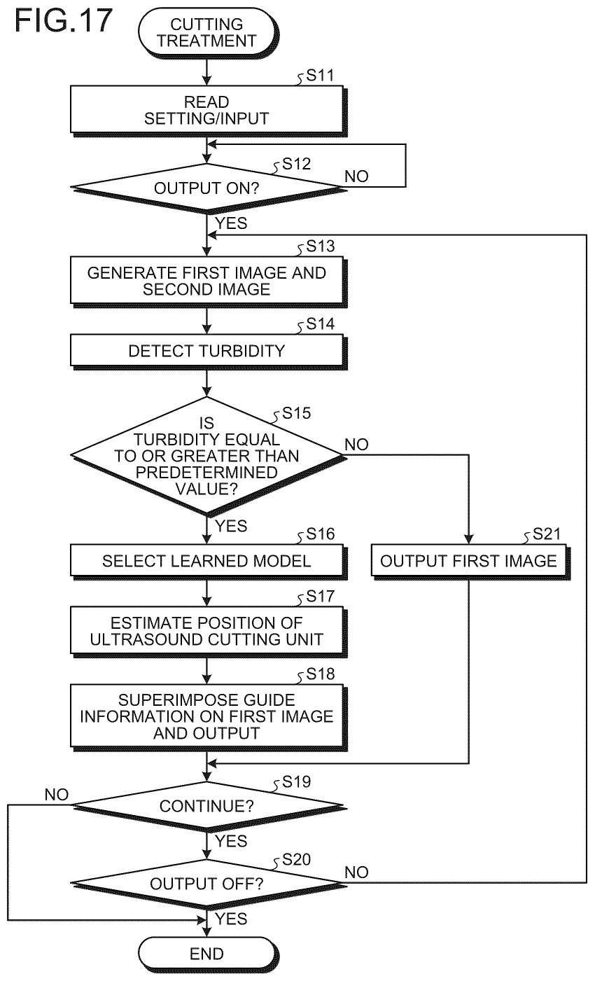

FIG. 17 is a diagram illustrating an outline of processing executed in the cutting treatment by the endoscope control device 202.

Note that, in the following description, it is assumed that each processing is executed according to the control of the CPU of each control device, but for example, any one of the control devices such as the network control device 7 may collectively execute the processing.

The CPU 227 communicates with each device, and performs setting of a control parameter for each of the treatment device 3 and the perfusion device 5 and input of the control parameter for each of the treatment device 3 and the perfusion device 5 (step S11).

Subsequently, the CPU 227 determines whether the devices of the respective units configuring the treatment system 1 are in the output ON state (step S12). When the CPU 227 determines that the devices of the respective units configuring the treatment system 1 are in the output ON state (step S12: Yes), the endoscope control device 202 proceeds to step S13 to be described later. On the other hand, when the CPU 227 determines that the devices of the respective units configuring the treatment system 1 are not in the output ON state (step S12: No), the CPU 227 continues the determination until the devices of the respective units configuring the treatment system 1 are in the output ON state.

Thereafter, the first image generation unit 2222a and the second image generation unit 2222b acquire image data from the imaging portion 204 and generate a first image and a second image (step S13).

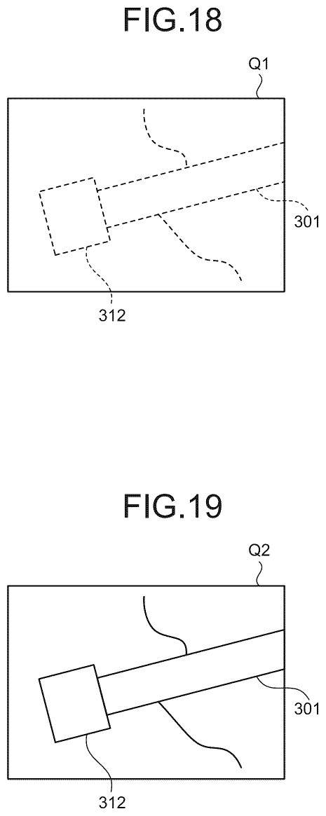

FIG. 18 is a diagram illustrating an example of the first image generated by the first image generation unit 2222a.

FIG. 19 is a diagram illustrating an example of the second image generated by the second image generation unit 2222b.

Note that, in FIGS. 18 and 19, a case of the first image and the second image in a state in which the visual field of the endoscope 201 is poor will be described.

That is, a case of image data (turbid image data) captured when turbidity occurs in the perfusate will be described.

As illustrated in FIG. 18, the first image generation unit 2222a generates a first image Q1 based on image data (three pieces of image data of red, green, and blue) captured with visible light by the endoscope 201. Here, the operator cannot grasp the position of the ultrasound cutting unit 312 from the first image Q1 due to turbidity of the perfusate.

On the other hand, as illustrated in FIG. 19, the second image generation unit 2222b generates a second image Q2 using invisible light that is infrared light based on image data obtained by capturing an image of an area that is the same as the visual field of the endoscope 201 and is the same as the first image Q1 and includes at least the ultrasound cutting unit 312. Here, since the second image generation unit 2222b captures an image with invisible light that is infrared light, the operator can grasp the contour of the ultrasound cutting unit 312 from the second image Q2 regardless of the turbidity of the perfusate, but since it is different from the actual situation, the operator cannot grasp the position of a living body, the degree of turbidity, and the like.

Subsequently, the turbidity detection unit 2229 detects the turbidity of the visual field of the endoscope 201 based on the first image generated by the first image generation unit 2222a (step S14). Specifically, the turbidity detection unit 2229 detects the turbidity of the visual field of the endoscope 201 using any one of the luminance, the saturation, and the contrast of the first image.

Thereafter, the turbidity determination unit 2230 determines whether the turbidity of the visual field of the endoscope 201 detected by the turbidity detection unit 2229 is equal to or greater than a predetermined value (step S15). The turbidity determination unit 2230 determines whether the turbidity component of the visual field of the endoscope 201 detected by the turbidity detection unit 2229 is equal to or greater than a predetermined value. When the turbidity determination unit 2230 determines that the turbidity component of the visual field of the endoscope 201 detected by the turbidity detection unit 2229 is equal to or greater than the predetermined value (step S15: Yes), the endoscope control device 202 proceeds to step S16 to be described later. On the other hand, when the turbidity determination unit 2230 determines that the turbidity component of the visual field of the endoscope 201 detected by the turbidity detection unit 2229 is not equal to or greater than the predetermined value (step S15: No), the endoscope control device 202 proceeds to step S21 to be described later.

In step S16, the estimation unit 2226 selects a learned model stored in the learned model memory 2225 based on a determination result input from the switching determination unit 2221.

Subsequently, the estimation unit 2226 estimates the position of the ultrasound cutting unit 312 from at least a partial area of the first image based on a switching signal from the switching determination unit 2221 and at least one of the first image generated by the first image generation unit 2222a and the second image generated by the second image generation unit 2222b (step S17).

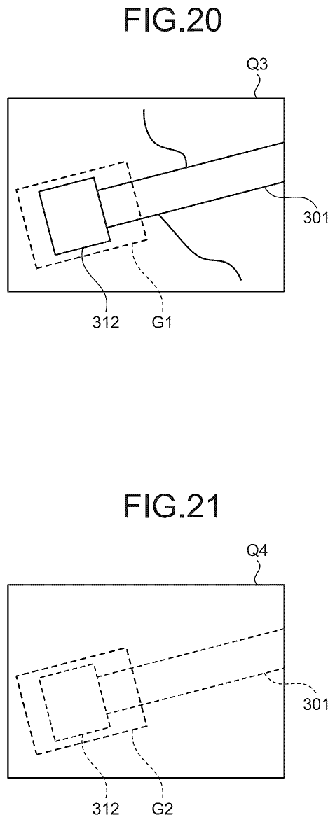

FIG. 20 is a diagram schematically illustrating an estimation result of a target object estimated by the estimation unit 2226.

As illustrated in FIG. 20, the estimation unit 2226 uses the switching signal and the second image as input data, and outputs an estimation result obtained by estimating a position or an area G1 of the ultrasound cutting unit 312 included in the second image Q3 as output data to the display image generation unit 2227.

Subsequently, the display image generation unit 2227 generates, based on the estimation result estimated by the estimation unit 2226, a display image in which guide information for guiding the position of the treatment instrument 301 appearing in the first image is superimposed on the first image, and outputs the display image to the display device 203 (step S18).

FIG. 21 is a diagram illustrating an example of the display image generated by the display image generation unit 2227.

As illustrated in FIG. 21, the display image generation unit 2227 generates a display image Q4 in which guide information G2 corresponding to the position or the area G1 of the ultrasound cutting unit 312 is superimposed on the first image Q1. As a result, even when the visual field of the endoscope 201 observing the treatment instrument 301 deteriorates due to cloudiness, the operator can perform cutting on the treatment target region 100 by the ultrasound cutting unit 312 without interruption because the guide information G2 is displayed by a frame in which the position of the ultrasound cutting unit 312, that is the distal end of the treatment instrument 301, is emphasized as compared with other areas.

In step S19, the CPU 227 determines whether the operator is continuing an operation on the subject. Specifically, the CPU 227 determines whether the treatment instrument control device 302 is supplying power to the treatment instrument 301, determines that the operator is continuing operation on the subject when the treatment instrument control device 302 is supplying power to the treatment instrument 301, and determines that the operator is not continuing operation on the subject when the treatment instrument control device 302 is not supplying power to the treatment instrument 301. When the CPU 227 determines that the operator is continuing the operation on the subject (step S19: Yes), the endoscope control device 202 proceeds to step S20 to be described later. On the other hand, when the CPU 227 determines that the operator is not continuing the operation on the subject (step S19: No), the endoscope control device 202 ends the present processing.

In step S20, the CPU 227 determines whether the devices of the respective units configuring the treatment system 1 are in the output OFF state. When the CPU 227 determines that the devices of the respective units configuring the treatment system 1 are in the output OFF state (step S20: Yes), the endoscope control device 202 ends the present processing. On the other hand, when the CPU 227 determines that the devices of the respective units configuring the treatment system 1 are not in the output OFF state (step S20: No), the endoscope control device 202 returns to step S13 described above.

In step S21, the CPU 227 performs normal control to output the first image to the endoscope control device 202. Specifically, the CPU 227 outputs the first image (color image) generated by the image processor 222 to the display device 203 to display the first image. As a result, the operator can perform an operation using the treatment instrument 301 while viewing the first image displayed on the display device 203. After step S21, the endoscope control device 202 proceeds to step S19.

### Outline of Learning by Learning Unit

Next, a content of a method of generating a learned model generated by the learning unit 2224 will be described.

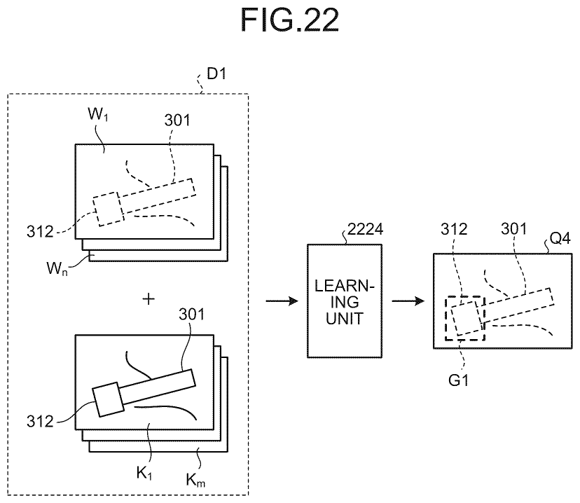

FIG. 22 is a diagram schematically illustrating the method of generating the learned model generated by the learning unit 2224.

As illustrated in FIG. 22, the learning unit 2224 generates the learned model in advance by performing machine learning using a plurality of pieces of image data generated by the endoscope device 2 as teacher data D1. As illustrated in FIG. 22, the teacher data is obtained by associating a plurality of treatment images W₁ to Wₙ (n=an integer of 2 or more) that are a plurality of pieces of treatment image data obtained by capturing an image of at least an area in which a living body is treated by the treatment instrument 301 that is an energy treatment instrument, in which the plurality of treatment images correspond to the treatment image data in which the visual field is poor due to bone meal or the like generated by the treatment, with a plurality of correction images K₁ to Kₘ(m=an integer of 2 or more) obtained by removing the turbidity from the treatment images W₁ to Wₙ by the image correction unit 2223, in which an annotation or a tag for the position of the area in which the living body is treated by the treatment instrument 301 and image processing parameters of the turbidity correction processing are applied. Although the above description is a case in which both the treatment images W₁ to Wₙ and the correction images K₁ to Kₘ are used, only one of the treatment images W₁ to Wₙ or the correction images K₁ to Kₘ may be used.

The learning unit 2224 performs machine learning using the teacher data D1, generates a learned model for outputting, as output data of an identification result, the position (coordinate address) of the area G1 in which the living body is treated by the treatment instrument 301, that is a target object in the image Q4 corresponding to the image data, with respect to the input image data, and records the learned model in the learned model memory 2225.

According to the embodiment described above, since the display image generation unit 2227 generates and outputs the display image Q3 based on the target object included in the first image estimated by the estimation unit 2226, it is possible to continuously perform the treatment of the treatment target region 100 by the treatment instrument 301 even when the visual field in the endoscope 201 deteriorates.

Furthermore, according to the embodiment, the display image generation unit 2227 generates and outputs the display image Q3 based on the estimation result of the target object included in either the first image or the second image estimated by the estimation unit 2226. As a result, since the operator can easily confirm the position of the ultrasound cutting unit 312, cutting of the treatment target region 100 by the ultrasound cutting unit 312 can be performed without interruption.

Note that, in the embodiment, the display image generation unit 2227 generates, based on the estimation result estimated by the estimation unit 2226, a display image in which the guide information for guiding the position of the treatment instrument 301 included in the first image is superimposed on the first image and outputs the display image to the display device 203, but the present disclosure is not limited thereto, and for example, the image correction unit 2223 may generate a display image using the first correction image obtained by correcting the turbidity (bone meal) of the first image based on the estimation result estimated by the estimation unit 2226 and may output the display image to the display device 203.

In addition, in the embodiment, the display image generation unit 2227 generates and outputs the display image Q3 based on the estimation result of the target object included in either the first image or the second image estimated by the estimation unit 2226, but a display image in which the guide information for guiding the position of the treatment instrument 301 included in the first image is superimposed on the first correction image corrected by the turbidity correction unit 2223a may be generated and output to the display device 203.

Further, in the embodiment, the estimation unit 2226 estimates the target object included in the second image using the learned model, but the present disclosure is not limited thereto, and a target object included in each of the first image, the first correction image, and the second correction image may be estimated.

### Modification

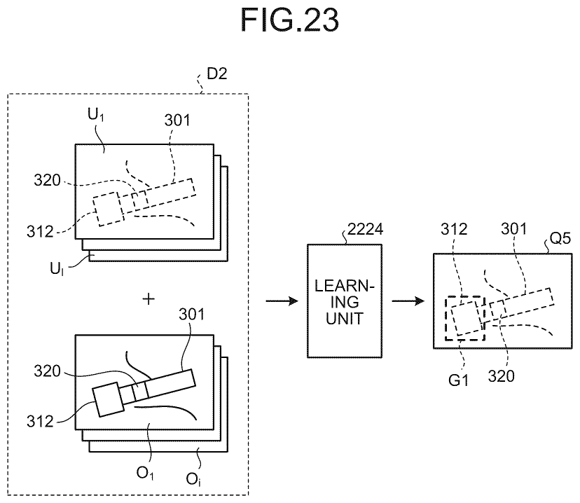

FIG. 23 is a diagram schematically illustrating a method of generating another learned model generated by the learning unit 2224 according to a modification of the embodiment.

As illustrated in FIG. 23, the learning unit 2224 may perform machine learning using, as teacher data D2, treatment images U₁ to Uₗ (l=an integer of 2 or more) that are a plurality of pieces of treatment image data obtained by capturing an image of at least an area in which a living body is treated by the treatment instrument 301 that is an energy treatment instrument, in which an index portion 320 provided in the treatment instrument 301 is included in the treatment images U₁ to U₁, and plurality of correction images O₁ to Oₗ (l=an integer of 2 or more) that are a plurality of correction images obtained by removing turbidity by the image correction unit 2223, in which an annotation or a tag for the index portion 320 of the treatment instrument 301 and image processing parameters of the turbidity correction processing are applied, and may generate, as output data, a learned model that outputs guide information G1 for guiding the position of an area including the ultrasound cutting unit 312 according to the position of the index portion 320 provided in the treatment instrument 301 included in the image Q5. Of course, the learning unit 2224 may generate, as output data, a learned model that outputs a movement amount of the index portion 320 provided in the treatment instrument 301 included in the image Q5 by performing machine learning using the teacher data D2. Here, the estimation unit 2226 estimates the position or the movement amount of the index portion as a target object in the first image using the learned model generated by the learning unit 2224 using the teacher data D2, and outputs the estimation result to the image correction unit 2223 and the display image generation unit 2227.

According to the modification of the embodiment described above, it is possible to output an image in which the position or movement of the treatment instrument 301 is identified while achieving the same effect as that of the embodiment described above.

Furthermore, in the modification of the embodiment, the learning unit 2224 may perform machine learning using a plurality of first images and a plurality of second images as teacher data, and may generate a learned model that outputs, as output data, a correction parameter of color information for correcting the second image that is infrared image into a color image. Here, the estimation unit 2226 estimates a correction parameter of the color information in the second image using the learned model generated by the learning unit 2224 using the teaching data configured by the plurality of first images and the plurality of second images, and outputs the estimation result to the image correction unit 2223 and the display image generation unit 2227. Then, the image correction unit 2223 corrects the second image that is infrared (monochrome) image into a color image based on the correction parameter of the color information of the estimation result estimated by the estimation unit 2226, and outputs the color image to the display image generation unit 2227. In addition to the color information, the estimation unit 2226 may estimate a parameter for correcting luminance information of the first image based on luminance information of the second image. As a result, even in the second image or when turbidity occurs in the first image, it is possible to display a color image in which the hue of the visual field of the endoscope 201 is reproduced in the second image. As a result, since the operator can easily confirm the position of the ultrasound cutting unit 312, cutting of the treatment target region 100 by the ultrasound cutting unit 312 can be performed without interruption.

### Other Embodiments

In addition, in the embodiment of the present disclosure, the treatment for turbidity caused by bone meal or the like in a liquid such as a perfusate has been described, but the present disclosure is not limited to in a liquid, and can be applied even in air. First to third embodiments can also be applied to deterioration in visibility in a visual field area of an endoscope due to cutting debris, fat mist, or the like caused by aerial treatment at a joint region.

Furthermore, in the embodiment of the present disclosure, the treatment in the knee joint has been described, but the present disclosure can be applied not only to the knee joint but also to other regions (spine and the like).

Furthermore, in the embodiment of the present disclosure, the present disclosure can also be applied to turbidity other than bone meal, and for example, can also be applied to debris such as soft tissue, synovial membrane, and fat, and other noise (cavitation of bubbles and the like). For example, the first to third embodiments can also be applied to turbidity or visual field deterioration caused by a cut piece of soft tissue such as cartilage, synovium, fat, or the like as a tissue piece as a factor of visual field deterioration caused by treatment by the treatment instrument 301.

In addition, in the embodiment of the present disclosure, the present disclosure can also be applied to deterioration in visual field due to fine bubbles generated by factors such as cavitation accompanying ultrasound vibration of the treatment instrument 301 in treatment in a liquid using the treatment instrument 301.

Furthermore, the embodiment of the present disclosure can also be applied to a case in which the visual field of the endoscope 201 is blocked by a relatively large tissue piece. Here, the endoscope control device 202 may determine whether the visual field of the endoscope 201 is shielded by a shielding object based on the first image, and may perform image processing of removing the shielding object using a known technique when it is determined that the visual field is shielded by the shielding object. Then, the endoscope control device 202 may perform image processing within a range not affecting the processing by using the size of a treatment area by the treatment instrument 301, the time during which the treatment target region 100 is shielded, and the like.

In addition, in the embodiment of the present disclosure, the present disclosure can also be applied when a filter capable of transmitting near infrared light (700 nm to 2500 nm) or an LED capable of emitting near infrared light is used instead of infrared light.

In the embodiment of the present disclosure, the learning unit 2224 performs machine learning using teacher data having a plurality of pieces of image data (a plurality of pieces of treatment image data) as the input parameters, but for example, the learning unit may perform machine learning to estimate a scene that occurs afterward based on a scene change.

Furthermore, in the embodiment of the present disclosure, the output of the estimation unit 2226 is not limited to correction necessity, and a data format and a content in a form that can be easily used by an external device, such as data for reconstructing an image, data including notification information, and codec data, may be output.

In addition, in the embodiment of the present disclosure, image data accompanied by cloudiness due to bone meal in the cutting treatment is used as the teacher data, but images including various types of turbidity generated in the cutting process, such as mist, blood, bone marrow fluid, and fat cut pieces in addition to the bone meal, can be used.

In addition, various embodiments can be formed by appropriately combining a plurality of components disclosed in the treatment system according to the embodiment of the present disclosure. For example, some components may be deleted from the entire components described in the treatment system according to the above-described first to third embodiments of the present disclosure. Furthermore, the components described in the treatment system according to the above-described first to third embodiments of the present disclosure may be appropriately combined with each other.

In addition, in the treatment system according to the embodiment of the present disclosure, the above-described “unit” can be replaced with “means”, “circuit”, or the like. For example, the control unit can be replaced with a control means or a control circuit.

In addition, the program to be executed by the treatment system according to the embodiment of the present disclosure is provided as file data in an installable format or an executable format by being stored in a computer-readable storage medium such as a CD-ROM, a flexible disk (FD), a CD-R, a digital versatile disk (DVD), a USB medium, or a flash memory.

In addition, the program to be executed by the treatment system according to the embodiment of the present disclosure may be stored on a computer connected to a network such as the Internet and may be provided by being downloaded via the network.

Note that, in the description of the flowcharts in the present specification, the context of processing between steps is clearly indicated using expressions such as “first”, “thereafter”, and “subsequently”, but the order of processing necessary for implementing the present disclosure is not uniquely determined by such expressions. That is, the order of processing in the flowcharts described in the present specification can be changed within a range without inconsistency. In addition, the program is not limited to such a program including simple branch processing, and more determination items may be comprehensively determined and branched.

According to the present disclosure, there is an effect that treatment by a treatment portion can be continuously performed even if a visual field of an endoscope deteriorates.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus comprising a processor comprising hardware, the processor being configured to:
estimate a target object in turbidity image data including turbidity generated when a living body is treated by an energy treatment instrument, from the turbidity image data that is input, using a learned model obtained by performing machine learning using teacher data obtained by associating annotation image data with an identification result of identifying the target object in the turbidity image data, the annotation image data having an annotation applied to the target object; and
generate a display image related to the target object based on the turbidity image data that is input and the estimated target object.

2. The image processing apparatus according to claim 1, wherein
the processor is further configured to:
generate, based on the estimated target object, correction image data obtained by correcting the turbidity image data,
generate the display image based on either the turbidity image data or the correction image data and the estimated target object.

3. The image processing apparatus according to claim 1, wherein
the processor is further configured to estimate either a position or a shape of the target object in a liquid in which a powdery material is diffused.

4. The image processing apparatus according to claim 1, wherein
the target object is a powdery material diffused in a liquid, and
the processor is further configured to estimate a position of the powdery material.

5. The image processing apparatus according to claim 1, wherein
the target object is an index portion provided in the energy treatment instrument, and
the processor is further configured to estimate a position of the index portion in a liquid in which a powdery material is diffused.

6. The image processing apparatus according to claim 1, wherein
the target object is an index portion provided in the energy treatment instrument, and
the processor is further configured to estimate a movement amount of the index portion.

7. The image processing apparatus according to claim 2, wherein
the processor is further configured to acquire infrared image data from an imaging element configured to receive invisible light including at least an infrared wavelength band.

8. The image processing apparatus according to claim 1, further comprising
a learned model memory configured to record a plurality of learned models each corresponding to each of a drive time of the energy treatment instrument, an electrical characteristic of the energy treatment instrument to the living body, and power supply to the energy treatment instrument, wherein
the processor is further configured to select one of the plurality of learned models recorded in the learned model memory based on at least one of the drive time, the electrical characteristic, and the power supply input from outside of the processor.

9. A treatment system comprising:
an energy treatment instrument; an imaging device; and an image processing apparatus,
wherein the energy treatment instrument includes
a treatment instrument main body portion extending from a proximal end side to a distal end side in a longitudinal direction of the energy treatment instrument, and
a treatment portion provided on a distal end side of the treatment instrument main body portion, the treatment portion being configured to treat a living body,
the imaging device includes
a casing main body configured to be inserted into a subject, the casing extending from a proximal end side to a distal end side in a longitudinal direction of the imaging device,
an illumination portion configured to emit illumination light toward at least an area in which the living body is treated by the energy treatment instrument, and
an imaging portion configured to generate turbidity image data including at least a part of an area in which the living body is treated by the energy treatment instrument and turbidity is generated, and
the image processing apparatus comprises a processor comprising hardware, the processor being configured to:
estimate a target object in the turbidity image data including the turbidity generated when the living body is treated by the energy treatment instrument, from the turbidity image data that is input, using a learned model obtained by performing machine learning using teacher data obtained by associating annotation image data with an identification result of identifying the target object in the turbidity image data, the annotation image data having an annotation applied to the target object; and
generate a display image related to the target object based on the turbidity image data that is input and the estimated target object.

10. A learning apparatus comprising
a processor comprising hardware, the processor being configured to generate a learned model by performing machine learning using teacher data, wherein
the teacher data uses, as input data, a plurality of pieces of treatment image data obtained by capturing an image of an area in which a living body is treated by an energy treatment instrument and a plurality of pieces of annotation image data to which an annotation of a target object included in a plurality of treatment images respectively corresponding to the plurality of pieces of treatment image data is applied, and outputs, as output data, an identification result in which the target object is identified, the target object being included in an image corresponding to image data including at least a part of the area in which the living body is treated by the energy treatment instrument.

11. The learning apparatus according to claim 10, wherein
the annotation image data is correction image data obtained by performing turbidity correction processing on each piece of the treatment image data and correction image data to which the annotation is applied.

12. The learning apparatus according to claim 10, wherein
the annotation image data is infrared image data acquired by an imaging element configured to receive invisible light including at least an infrared wavelength band and infrared image data to which the annotation is applied.

13. The learning apparatus according to claim 10, wherein
the treatment image data is image data obtained by capturing an image of a liquid in which a powdery material is diffused by treating the living body with the energy treatment instrument.

14. The learning apparatus according to claim 13, wherein
the annotation is a position of an index portion provided in the energy treatment instrument included in an image corresponding to the image data.

15. The learning apparatus according to claim 13, wherein
the annotation is a movement amount of an index portion provided in the energy treatment instrument included in an image corresponding to the image data.

16. The learning apparatus according to claim 10, wherein
the processor is further configured to
set, as the input data, one or more of a drive time of the energy treatment instrument, an electrical characteristic of the energy treatment instrument to the living body, and power supply to the energy treatment instrument, and
generate the learned model of each of the drive time, the electrical characteristic, and the power supply.

17. An image processing method executed by an image processing apparatus comprising a processor comprising hardware, the method comprising:
estimating a target object in turbidity image data including turbidity generated when a living body is treated by an energy treatment instrument, from the turbidity image data that is input, using a learned model obtained by performing machine learning using teacher data obtained by associating annotation image data with an identification result of identifying the target object in the turbidity image data, the annotation image data having an annotation applied to the target object; and
generating a display image related to the target object based on the turbidity image data that is input and the estimated target object.
